(19) United States
(12) Patent Application Publication
(10) Pub. No.: US 2026/0284110 A1
(43) Pub. Date: Sep. 24, 2026

(54) INVARIANT NATURAL KILLER T CELLS FOR TREATING CANCER

(71) Applicant: MiNK Therapeutics, Inc., New York, NY (US)

(72) Inventor: Marc van Dijk, Bosch en Duin (NL)

(73) Assignee: MiNK Therapeutics, Inc., New York, NY (US)

(21) Appl. No.: 19/476,442

(22) PCT Filed: Apr. 18, 2024
(86) PCT No.: PCT/US2024/025189

§ 371 Date: Oct. 17, 2025

Related U.S. Application Data
(60) Provisional application No. 63/460,257, filed on Apr. 18, 2023.

(51) Int. Cl. A61K 35/17; A61K 39/00; A61K 39/395; A61P 35/00

(52) U.S. Cl. CPC A61K 35/17; A61K 39/3955; A61P 35/00; A61K 2039/505; A61K 2039/545

(57) ABSTRACT

The present disclosure relates to compositions comprising invariant natural killer T (iNKT) cells, and methods of using the compositions comprising the iNKT cells for treating cancer.

### RELATED APPLICATIONS

This application claims priority under 35 U.S.C. § 119 (e) to U.S. Provisional Application Ser. No. 63/460,257, filed Apr. 18, 2023, the entire contents of which are incorporated herein by reference.

### BACKGROUND

Invariant natural killer T (iNKT) cells are a unique subset of T cells that exhibit both direct and indirect anti-tumor activities. iNKT cells can recognize and kill tumor cells directly through the release of cytotoxic molecules, such as perforin and granzyme B, and by inducing apoptosis through the Fas/FasL pathway. Additionally, iNKT cells can act indirectly by stimulating other immune cells, such as dendritic cells, natural killer (NK) cells, and CD8+ T cells, to target and kill tumor cells. iNKT cells also play a role in regulating the immune response by producing cytokines, such as interferon-gamma and interleukin-4, that promote the activation and differentiation of other immune cells.

### SUMMARY

An allogeneic iNKT cell product called agenT-797 was developed. This cell product was ex vivo expanded and has the potential to treat a broad spectrum of diseases, including solid tumors. A Phase 1 clinical trial to assess the safety and efficacy of agenT-797 as a monotherapy or in combination with pembrolizumab or nivolumab in patients with relapsed or refractory solid tumors (NCT05108623) was conducted.

The present disclosure, at least in part, relates to compositions comprising allogeneic invariant natural kill T (iNKT) cells (e.g., unmodified iNKT cells), and methods of using the compositions comprising the iNKT cells for treating cancer.

Aspects of the disclosure relate to compositions and methods for treating certain cancers, for example relapsed/refractory solid tumor cancers.

Accordingly, in some aspects, the disclosure provides a method for treating a relapsed/refractory solid tumor cancer, the method comprising administering to a subject in need thereof a composition comprising at least 95% allogeneic invariant natural killer cells (iNKTs). In some embodiments, the iNKT cells are isolated from a single donor. In some embodiments, the iNKT cells are isolated from two or more donors.

In some aspects, the disclosure provides a method for treating a relapsed/refractory solid tumor cancer, the method comprising administering to a subject in need thereof a composition comprising at least 95% allogeneic invariant natural killer cells (iNKTs) (e.g., isolated from a single donor or two or more donors); and an immune checkpoint inhibitor (ICI).

In some aspects, the disclosure provides a method for treating gastric cancer in a subject in need thereof, the method comprising administering to the subject a composition comprising at least 95% allogeneic invariant natural killer cells (iNKTs) (e.g., isolated from a single donor or two or more donors); and an immune checkpoint inhibitor (ICI).

In some embodiments, a relapsed/refractory solid tumor cancer is a gastric cancer, lung cancer, or testicular cancer. In some embodiments, a relapsed/refractory solid tumor cancer is an ampullary cancer, appendiceal cancer, biliary duct cancer, cholangiocarcinoma, colorectal cancer (CRC), duodenal cancer, non-small cell lung carcinoma (NSCLC), ocular melanoma, pancreatic cancer, or prostate cancer.

In some embodiments, a subject has been previously administered one or more of the following agents: nivolumab, pembrolizumab, botensilimab, FOLFOX, oxaliplatin, leucovorin, fluorocil, bleomycin, etopidide, cisplatin, vinblasine, ifofamise, carboplatin, ipilumumab, CD1D RM 684, or an autologous stem cell transplant. In some embodiments, a subject has not previously been administered an anti-CD1d monoclonal antibody. In some embodiments, administration does not comprise toxic lymphodepletion of the subject. In some embodiments, a subject does not have an active autoimmune disorder.

In some embodiments, from about 4.3×10⁶ to about 1.4×10⁷ iNKT cells/kg are administered to a subject. In some embodiments, a subject has gastric cancer and is administered about 4.3×10⁶ iNKT cells/kg. In some embodiments, a subject has testicular cancer and is administered about 1.4×10⁷ iNKT cells/kg.

In some embodiments, methods described herein further comprise administering one or more immune checkpoint inhibitors (ICI) to a subject. In some embodiments, one or more ICI comprises nivolumab, pembrolizumab, and/or botensilimab. In some embodiments, the methods comprise administering 200 mg of nivolumab to a subject.

In some embodiments, after administration of iNKTs to a subject, a level of one or more pro-inflammatory cytokines is increased in the subject relative to the level of the one or more pro-inflammatory cytokine in the subject prior to the administration. In some embodiments, one or more pro-inflammatory cytokine is IFNγ or TNFα.

In some embodiments, after administration of iNKTs to a subject, a level of immune infiltration of the subject's TME is increased relative to the level of immune infiltration of the TME prior to the administration.

In some embodiments, after administration of iNKTs to a subject, an expression level of one or more of the following genes is increased in the subject relative to the expression level of the one or more genes prior to the administration: GZMB, GZMH, GZMA, GZMK, GZMM, GNLY, PRF1, LEF1, TRAIL, FASLG, LAMP1, NKG7, CD25, CD69, 4-1BB, HLA-DRA, CD44, TCF1, TBET, EOMES, CCL3, CCL4, IFNG, TNF, DNAM1, 2B4, SLAMF6, NKG2D, NKG2C, NKG2A, or CD56.

In some embodiments, administration of iNKTs to a subject results in inhibition of growth or shrinkage of a tumor in the subject. In some embodiments, a subject is a human.

### BRIEF DESCRIPTION OF THE DRAWINGS

The accompanying drawings, which are incorporated in and constitute a part of this specification, illustrate certain embodiments, and together with the written description, serve to provide non-limiting examples of certain aspects of the compositions and methods disclosed herein.

FIG. 1 is a schematic depicting iNKT cell anti-cancer mechanisms.

FIG. 2 is the study design for agenT-797 in solid tumors.

FIGS. 3A-3B show pro-inflammatory cytokine signature. FIG. 3A shows pre- and post-infusion serum levels of select pro-inflammatory and anti-inflammatory cytokines over the first month following infusion (n=30). Data grouped into three timepoints: day 1 pre-infusion (Pre; single timepoint), early post-infusion window (0.5 hours post infusion to day 8; D1-D8), and a late post-infusion time window (days 15-29; D15-29). Upper limit of cytokines in healthy people as published in the literature: IFNγ: 25 pg/ml; TNFα: 30 pg/ml; IL-1β: 12 pg/ml; IL-2:20 pg/ml; IL-4:1 pg/ml; IL-6:25 pg/ml. Upper limit indicated as dotted line on graphs where possible. Significant changes post dosing are indicated with asterisks (**p<0.01; ***p<0.001; Analysis using non-parametric Friedman test). The most significant early post-dosing change was observed for IFNγ. FIG. 2B shows IFNγ data for each timepoint. IFNγ release is a hallmark of iNKT activation, and increased detection in serum may indicate iNKT activation at tumor site. No corresponding increase in IFNγ or TNFα levels has been observed in our trial with agenT-797 in COVID-19/ARDS, where an anti-inflammatory signature predominates.

FIGS. 4A-4B show persistence of agenT-797 in the periphery. Quantification of agenT-797 in patient PBMC by digital droplet PCR (ddPCR) based on single nucleotide polymorphisms (SNPs) unique to donor material. FIG. 4A shows average persistence measurements for DL1 (Red), DL2 (Blue) and total dataset (Black) highlighting peak of agenT-797 levels in the periphery on D2 post-infusion, which scales with dose level. Low level persistence in blood detected up to week 8 post-infusion. FIG. 4B shows the observed decline of agenT-797 levels in blood post D2 is consistent with the dynamics of blood-to-tissue distribution of agenT-797 observed in vivo in a previously described murine xenograft. Analysis of tissue persistence of agenT-797 in patients is currently in-process using cell-free DNA (cfDNA) analysis (readout Q2 2023).

FIGS. 5A-5B show the change in baseline scans in patients who received agenT-797 and have baseline as well as at least one post-baseline measure of target lesions. FIG. 5A shows the percent target lesion change form baseline. FIG. 5B shows percent target lesions change from baseline over time.

FIGS. 6A-6B show responses in patients treated with agenT-797+nivolumab. FIG. 6A are scans from MSI-high gastric cancer patient with partial response. FIG. 5B show characteristics of patients with response to combination therapy.

FIG. 7 shows multiplex immunofluorescence (mIF) on FFPE tumor sections from a responding gastric cancer patient. On-treatment biopsy demonstrates dramatic infiltration of tumor by T cells. Infiltrate consists predominantly of CD8+ cytotoxic T lymphocytes (CTL), with Ki-67 biomarker expression indicative of recent CTL activation and cell division. (images analyzed by automated image analysis as shown in FIG. 8)

FIG. 8 shows profiling tumor and immune cells in gastric cancer patient with partial response. Gastric cancer patient shown in red, all other patients shown in black. (Left to right, top to bottom) High mutational burden correlated with a high neoantigen score. Tumor infiltrating T cells demonstrated a high degree of clonality, which increased following treatment with agenT-797. TCR CDR3 sequence analysis indicates a post-treatment switch in the predominant TCR clone, with infusion of agenT-797 followed by an expansion of a minority pre-existent T cell clone (red open circles, dotted line). This increase of a specific T cell clone corresponds with markers of T cell activation and proliferation as measured by mIF and automated image analysis, indicating intra-tumoral expansion of mainly CTL following treatment with agenT-797. Taken together, high tumor mutational burden leads to increased neoantigen presentation which is only partially effective at driving clonal expansion of anti-tumor T cells, even in the presence of anti PD-1 checkpoint inhibitors. Following administration of agenT-797 systemic and local TH-1 signatures are detected, which lead to enhanced immune infiltration of tumor and relieves the blockage of neoantigen driven expansion of anti-tumor CTL. WES/RNAseq data (top 4 graphs) from 10 patients and mIF data (bottom 3 graphs) from 16 patients.

FIGS. 9A-9D show RNAseq gene expression analysis of screening (left bars) and on-treatment (onTx) (right bars) tumor biopsies from gastric cancer patient. On-treatment tumor material (D15) exhibits signature of increased tumor immune infiltration and activity. On-treatment gene expression signatures indicate increased (FIG. 9A) cytotoxic activity, (FIG. 9B) immune cell activation, and (FIG. 9C) TH-1 polarization, and are consistent with the infiltration of tumor by cytotoxic lymphocytes including CTL, (FIG. 9D) NK cells or iNKT cells. Gene expression shown as transcripts per million (TPM).

### DETAILED DESCRIPTION

The present disclosure, at least in part, relates to compositions comprising allogeneic invariant natural kill T (iNKT), and methods of using the compositions comprising the iNKT cells for treating cancer.

The foregoing and other aspects, implementations, acts, functionalities, features and embodiments of the present teachings can be more fully understood from the following description in conjunction with the accompanying drawings.

I. Therapeutic Treatments Using Invariant Natural Killer T (iNKT) Cells

In some aspects, the present disclosure provides a method of treating a subjecting having a viral infection, the method comprising administering the subject a composition comprising invariant natural killer T (iNKT) cells. In some embodiments, the present disclosure contemplates the use of a composition comprising iNKT cells (e.g., unmodified, allogenic iNKT cells) in a method for treating cancer (e.g., refractory/relapsed cancer). As used herein, the terms “administering” or “administration” means to provide a therapeutic agent (e.g., iNKT cells) or a composition thereof (e.g., a composition comprising iNKT cells) to a subject in a manner that is physiologically and/or pharmacologically useful (e.g., to treat a disease or a symptom or complication associated with the disease in the subject). As used herein, the term “subject” refers to a mammal. In some embodiments, a subject is a human. In some embodiments, a subject is a patient, e.g., a human patient that has or is suspected of having a disease. In some embodiments, the subject is a human patient who has cancer. As used herein, the term “treating” or “treatment” refers to the application or administration of a composition including one or more active agents (e.g., unmodified, allogeneic iNKT cells) to a subject who has cancer, or a predisposition of cancer, with the purpose to cure, heal, alleviate, relieve, alter, remedy, ameliorate, improve, or affect the cancer. Alleviating cancer includes delaying or preventing the development or progression of the disease, reducing disease severity, reducing tumor volume, and/or promoting survival.

In some aspects, the present disclosure also provides a method for treating a subject having cancer. As used herein “relapsed/refractory cancer” refers to a cancer has recurred or that did not respond to treatment or stopped responding during the course of treatment. As used herein “cancer” refers to a group of diseases characterized by transformation of cells to a state where the cells divide uncontrollably and have the ability to infiltrate and destroy normal body tissue. In some embodiments, a cancer is a solid tumor cancer. Solid tumor cancers are characterized by development of tumors (e.g., masses of abnormal, cancerous cells) that generally do not contain cysts or liquid. Examples of solid tumors include but are not limited to sarcomas, carcinomas, and lymphomas.

In some embodiments, the present disclosure provides compositions and methods for treating solid tumor cancer (e.g., relapsed/refractory solid tumor cancer) in a subject. In some embodiments, a subject has gastric cancer, lung cancer, or testicular cancer. In some embodiments, a subject has lung cancer, and the lung cancer is a non-small cell lung cancer. Non-small cell Lung Cancer (NSCLC) is a solid tumor cancer and the most common type of lung cancer. NSCLC includes large cell lung adenocarcinoma and squamous cell carcinoma of the lung. In some embodiments, a subject has NSCLC that is refractory to existing treatment (e.g., immune check point inhibitor treatment). In some embodiments, a subject has gastric cancer. Gastric cancer, also referred to as stomach cancer, is a solid tumor cancer that develops in cells of the stomach lining. In some embodiments, a subject has gastric cancer that is refractory to existing treatment (e.g., immune check point inhibitor treatment). In some embodiments, a subject has testicular cancer. Testicular cancer is a solid tumor cancer that begins in the cells of the testicles. In some embodiments, a subject has testicular cancer that is refractory to existing treatment (e.g., immune check point inhibitor treatment).

In some embodiments, the tumor microenvironment (TME) of a solid tumor (e.g., refractory and/or relapsed solid tumor) is immunosuppressive. Tumor microenvironment (TME): As used herein, the term “tumor microenvironment” or “TME” refers to the fluid, molecules, cells, and/or tissues around and/or at the tumor site. The TME can include normal cells, tumor cells, tumor stromal cells (e.g., stromal fibroblasts), blood vessels, blood, immune cells, and the non-cellular components (e.g., extracellular matrix such as collagen, fibronectin, hyaluronan, laminin, and secreted molecules such as cytokines, etc.). In some embodiments, TME plays an important role in tumor development, antitumor immunity, and response to antitumor therapy in solid tumor (see, e.g., Chaudhuri et al., (2018), Mechanobiology of tumor growth. Chemical Reviews, 118 (14), 6499-6515). In some embodiments, TME poses as a barrier to tumor infiltration by T cells. In some embodiments, the TME barrier affects the efficacy of cancer immunotherapy (e.g., adoptive T cell therapy, and/or immune checkpoint inhibitor therapy). In some embodiments, in solid tumor TME, there is increased deposition of fibrillar matrix components (such as collagen and fibronectin, and/or an increase in protein crosslinking as well as linearization, which leading to increased stiffness in the tumor relative to the corresponding healthy tissue. In some embodiments, the activity of tumoral stromal/mesenchymal cells, fibroblasts, and tumor-associated macrophages (TAMs) favors tumor proliferation. In some embodiments, TME restricts the access for immune cells and drugs into the tumor, rendering treatment ineffective. Typically, tumor rejection starts with the infiltration of the tumor cells by immune cells including T cells (e.g., cytotoxic T lymphocyte (CTL)) response. In some embodiments, migration and infiltration of T cells is significantly reduced in tumor TME. In some embodiments, solid tumor TME facilitates the development of resistance to cancer immunotherapy (e.g., adoptive T cell therapy such as CAR T cell therapy and/or immune checkpoint inhibitor (ICI) therapy).

In some embodiments, a subject having cancer (e.g., gastric cancer, lung cancer, or testicular cancer) has a cancer that is HER-2 negative. In some embodiments, a subject having cancer (e.g., gastric cancer, lung cancer, or testicular cancer) has a cancer that is PD-L1 positive. In some embodiments, a subject having gastric cancer that is HER-2 negative and PD-L1 positive. In some embodiments, a subject having gastric cancer has a PD-L1 combined positivity score (CPS) of at least 1, at least 2, at least 3, at least 4, at least 5, at least 6, at least 7, at least 8, at least 9, at least 10, or higher. In some embodiments, a subject having gastric cancer has a PD-L1 combined positivity score (CPS) in the range of 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10. The PD-L1 combined positive score (CPS) is the proportion of PD-L1-positive cells among all tumor cells (see, e.g., Chong et al., Tracking circulating PD-L1-positive cells to monitor the outcome of patients with gastric cancer receiving anti-HER2 plus anti-PD-1 therapy, Human Cell 37, 258-270 (2024)). CPS can be measured using any suitable methods. In some embodiments, a subject has gastric cancer, and is HER-2 negative and PD-L1 positive with a CPS score of 7.

In some embodiments, a subject having cancer (e.g., gastric cancer, lung cancer, or testicular cancer) has high microsatellite instability (MSI-H) (e.g., as indicated by positive expression of MSH-2 and MSH-6). In some embodiments, a subject has MSI-H has high tumor mutational burden relative to a subject that has the sane caner but without MSI-H. In some embodiments, a subject having MSI-H cancer (e.g., gastric cancer, lung cancer, or testicular cancer) has a mutational burden of at least 25 mut/MB, at least 30 mut/MB, at least 35 mut/MB, at least 40 mut/MB, at least 45 mut/MB, at least 50 mut/MB, at least 55 mut/MB, at least 60 mut/MB, at least 65 mut/MB, at least 70 mut/MB, at least 75 mut/MB, at least 80 mut/MB, at least 85 mut/MB, at least 90 mut/MB, at least 95 mut/MB, at least 100 mut/MB, at least 150 mut/MB, at least 200 mut/MB, at least 300 mut/MB, at least 400 mut/MB, at least 500 mut/MB, or more. In some embodiments, a subject having MSI-H cancer (e.g., gastric cancer, lung cancer, or testicular cancer) has a mutational burden in the range of 25-500 mut/MB, 25-400 mut/MB, 25-300 mut/MB, 25-200 mut/MB, 25-150 mut/MB, 25-125 mut/MB, 25-100 mut/MB, 25-75 mut/MB, 25-50 mut/MB, 50-500 mut/MB, 50-400 mut/MB, 50-300 mut/MB, 50-200 mut/MB, 50-150 mut/MB, 50-125 mut/MB, 50-100 mut/MB, at least 50-75 mut/MB, 75-500 mut/MB, 75-400 mut/MB, 75-300 mut/MB, 75-200 mut/MB, 75-150 mut/MB, 75-125 mut/MB, 75-100 mut/MB, 75-80 mut/MB, 75-85 mut/MB, 80-100 mut/MB, 80-90 mut/MB, 80-85 mut/MB, 80-120 mut/MB, 80-150 mut/MB, 80-200 mut/MB, 80-300 mut/MB, 80-400 mut/MB, 80-500 mut/MB, 100-500 mut/MB, 100-400 mut/MB, 100-300 mut/MB, 100-200 mut/MB, 100-150 mut/MB, 100-125 mut/MB, 200-500 mut/MB, 200-400 mut/MB, 200-300 mut/MB, 200-250 mut/MB, 300-500 mut/MB, 300-400 mut/MB, or 400-500 mut/MB. In some embodiments, a subject having cancer with high tumor mutation burden in the subject has increased neoantigen presentation, which leads to ineffective T cell clonal expansion. In some embodiments, ineffective T cell clonal expansion leads to insufficient immune response to tumor cells. In some embodiments, a subject having cancer (e.g., gastric cancer) with a CPS score of at least one and MSI-H is suitable for immune checkpoint inhibitor (ICI) therapy. Accordingly, in some embodiments, a subject has previously received ICI therapy as treatment for the cancer. In some embodiments, a subject the ICI therapy includes but are not limited to nivolumab, pembrolizumab, or botensilimab. In some embodiments, a subject also previously received other anti-cancer therapy such as FOLFOX, oxaliplatin, leucovorin, fluorocil, bleomycin, etopidide, cisplatin, vinblasine, ifofamise, carboplatin, ipilumumab, CD1D RM 684, or an autologous stem cell transplant. In some embodiments, after treatments (e.g., 1 treatment, 2 treatments, 3 treatments, 4 treatments, 5 treatments, 6 treatments, 7 treatments, 8 treatments, 9 treatments, 10 treatments, 11 treatments, 12 treatments, 13 treatments, 14 treatments, 15 treatments, 16 treatments, 17 treatments, 18 treatments, 19 treatments, 20 treatments, 21 treatments, 22 treatments, 23 treatments, 24 treatments, 25 treatments, or more) of ICI therapy, the subject acquired resistance to one or more ICI therapy. In some embodiments, a subject was previously administered pembrolizumab but developed resistance to it. In some embodiments, a subject having resistance to pembrolizumab was switched to nivolumab, but later also developed resistance to nivolumab. In some embodiments, resistance to ICI therapy is evidenced by partial response to the treatment (e.g., partial T cell clonal expansion in the tumor) and progress of the cancer itself (e.g., increased tumor burden and/or tumor metastasis). In some embodiments, a subject has gastric cancer (e.g., HER-2 negative and PD-L1 positive with MSI-H acquired resistance to one or more ICI therapy (e.g., pembrolizumab and/or nivolumab) shows worsening of gastric wall thickening and/or progression of omental nodularity (i.e., modules in omentum) relative to prior to the ICI treatments.

The present disclosure, at least in part, relates to the discovery that administration of iNKT cell (e.g., unmodified, allogenic iNKT cells isolated from a single donor or two or more donors) compositions (e.g., agenT-797) to a subject having cancer (e.g., gastric cancer, lung cancer, or testicular cancer) that is resistant to ICI therapy can overcome resistance to ICI therapy. In some embodiments, a subject does not have an active autoimmune disorder. In some embodiments, subject has not previously received anti-CD1d monoclonal antibody.

In some embodiments, a subject does not undergo lymphodepletion prior to, or concurrent with, treatment with iNKT cell or composition thereof (e.g., agenT-797). Lymphodepletion is frequently performed prior to immunotherapies, such as adoptive cell therapy such as CAR T therapies. In some instances, subject receiving adoptive cell therapy receive a course of chemotherapy to deplete the T cells from the subject, for instance, to debulk a tumor, alter tumor phenotype, modify the tumor microenvironment, remove cytokine sinks (e.g., make IL-2, IL-7, and IL-15 more available) and suppress the host immune system. In some instances, lymphodepletion can effectively prolong the persistence of infused cells and increase the effectiveness of treatment. However, lymphodepletion has multiple negative effects including, neutropenia, anemia, thrombocytopenia, and immunosuppression, and toxicities associated with lymphodepletion agents such as fludarabine and cyclophosphamide. In some embodiments, a subject receiving iNKT cell therapy (e.g., agenT-797) does not need lymphodepletion therapy prior to administration of the iNKT cells. In certain embodiments, the subject does not receive treatment with, fludarabine, or cyclophosphamide prior to, or concurrent with, administration of the iNKT cells or a composition thereof (e.g., agenT-797). In some embodiments, iNKT cell therapy (e.g., agenT-797) without lymphodepletion are effective in reducing tumor burden and/or improve survival. In some embodiments, iNKT cell therapy (e.g., agenT-797) without lymphodepletion exhibit long term efficacy in killing cancer cells. In some embodiments, iNKT cell therapy (e.g., agenT-797) without lymphodepletion does not induce graft-versus-host disease (GVHD).

In some embodiments, a subject is administered a composition comprising at least 90%, at least 91%, at least 92%, at least 93%, at least 94%, at least 95%, at least 96%, at least 97%, at least 98%, at least 99% iNKT cells (e.g., isolated from a single donor or two or more donors). In some embodiments, a subject is administered at least 1×10⁶ cells/kg, at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, at least 1×10⁷ cells/kg, at least 2×10⁷ cells/kg, at least 3×10⁷ cells/kg, at least 4×10⁷ cells/kg, at least 5×10⁷ cells/kg, or more. In some embodiments, a subject is administered iNKT cells in the range of 1×10⁶ to 5×10⁷ cells/kg, 1×10⁶ to 4×10⁷ cells/kg, 1×10⁶ to 3×10⁷ cells/kg, 1×10⁶ to 2×10⁷ cells/kg, 1×10⁶ to 1×10⁷ cells/kg, 1×10⁶ to 9×10⁶ cells/kg, 1×10⁶ to 8×10⁶ cells/kg, 1×10⁶ to 7×10⁶ cells/kg, 1×10⁶ to 6×10⁶ cells/kg, 1×10⁶ to 5×10⁶ cells/kg, 1×10⁶ to 4×10⁶ cells/kg, 1×10⁶ to 3×10⁶ cells/kg, 1×10⁶ to 2×10⁶ cells/kg, 2×10⁶ to 5×10⁷ cells/kg, 2×10⁶ to 4×10⁷ cells/kg, 2×10⁶ to 3×10⁷ cells/kg, 2×10⁶ to 2×10⁷ cells/kg, 2×10⁶ to 1×10⁷ cells/kg, 2×10⁶ to 9×10⁶ cells/kg, 2×10⁶ to 8×10⁶ cells/kg, 2×10⁶ to 7×10⁶ cells/kg, 2×10⁶ to 6×10⁶ cells/kg, 2×10⁶ to 5×10⁶ cells/kg, 2×10⁶ to 4×10⁶ cells/kg, 2×10⁶ to 3×10⁶ cells/kg, 3×10⁶ to 5×10⁷ cells/kg, 3×10⁶ to 4×10⁷ cells/kg, 3×10⁶ to 3×10⁷ cells/kg, 3×10⁶ to 2×10⁷ cells/kg, 3×10⁶ to 1×10⁷ cells/kg, 3×10⁶ to 9×10⁶ cells/kg, 3×10⁶ to 8×10⁶ cells/kg, 3×10⁶ to 7×10⁶ cells/kg, 3×10⁶ to 6×10⁶ cells/kg, 3×10⁶ to 5×10⁶ cells/kg, 3×10⁶ to 4×10⁶ cells/kg, 4×10⁶ to 5×10⁷ cells/kg, 4×10⁶ to 4×10⁷ cells/kg, 4×10⁶ to 3×10⁷ cells/kg, 4×10⁶ to 2×10⁷ cells/kg, 4.3×10⁶ to 1.7×10⁷ cells/kg, 4×10⁶ to 1×10⁷ cells/kg, 4×10⁶ to 9×10⁶ cells/kg, 4×10⁶ to 8×10⁶ cells/kg, 4×10⁶ to 7×10⁶ cells/kg, 4×10⁶ to 6×10⁶ cells/kg, 4×10⁶ to 5×10⁶ cells/kg, 5×10⁶ to 5×10⁷ cells/kg, 5×10⁶ to 4×10⁷ cells/kg, 5×10⁶ to 3×10⁷ cells/kg, 5×10⁶ to 2×10⁷ cells/kg, 5×10⁶ to 1×10⁷ cells/kg, 5×10⁶ to 9×10⁶ cells/kg, 5×10⁶ to 8×10⁶ cells/kg, 5×10⁶ to 7×10⁶ cells/kg, 5×10⁶ to 6×10⁶ cells/kg, 6×10⁶ to 5×10⁷ cells/kg, 6×10⁶ to 4×10⁷ cells/kg, 6×10⁶ to 3×10⁷ cells/kg, 6×10⁶ to 2×10⁷ cells/kg, 6×10⁶ to 1×10⁷ cells/kg, 6×10⁶ to 9×10⁶ cells/kg, 6×10⁶ to 8×10⁶ cells/kg, 6×10⁶ to 7×10⁶ cells/kg, 7×10⁶ to 5×10⁷ cells/kg, 7×10⁶ to 4×10⁷ cells/kg, 7×10⁶ to 3×10⁷ cells/kg, 7×10⁶ to 2×10⁷ cells/kg, 7×10⁶ to 1×10⁷ cells/kg, 7×10⁶ to 9×10⁶ cells/kg, 7×10⁶ to 8×10⁶ cells/kg, 8×10⁶ to 5×10⁷ cells/kg, 8×10⁶ to 4×10⁷ cells/kg, 8×10⁶ to 3×10⁷ cells/kg, 8×10⁶ to 2×10⁷ cells/kg, 8×10⁶ to 1×10⁷ cells/kg, 8×10⁶ to 9×10⁶ cells/kg, 9×10⁶ to 5×10⁷ cells/kg, 9×10⁶ to 4×10⁷ cells/kg, 9×10⁶ to 3×10⁷ cells/kg, 9×10⁶ to 2×10⁷ cells/kg, 9×10⁶ to 1×10⁷ cells/kg, 1×10⁷ to 5×10⁷ cells/kg, 1×10⁷ to 4×10⁷ cells/kg, 1×10⁷ to 3×10⁷ cells/kg, 1×10⁷ to 2×10⁷ cells/kg, 2×10⁷ to 5×10⁷ cells/kg, 2×10⁷ to 4×10⁷ cells/kg, 2×10⁷ to 3×10⁷ cells/kg, 3×10⁷ to 5×10⁷ cells/kg, 3×10⁷ to 4×10⁷ cells/kg, or 4×10⁷ to 5×10⁷ cells/kg. In some embodiments, a subject has gastric and is administered about 4.3×10⁶ iNKT cells/kg. In some embodiments, a subject has testicular cancer and is administered about 1.4×10⁷ iNKT cells/kg.

In some aspects, the present disclosure provides composition and methods for treating a relapsed/refractory solid tumor cancer (e.g., lung cancer, gastric cancer, testicular cancer). In some embodiments, the method comprising administering to a subject in need thereof: (i) a composition comprising at least 95% allogeneic invariant natural killer cells (iNKTs); and (ii) an immune checkpoint inhibitor (ICI) (e.g., nivolumab, pembrolizumab, botensilimab or any known ICI therapy). In some embodiments, the ICI is nivolumab. In some embodiments, a subject is administered a composition comprising at least 95% allogeneic invariant natural killer cells (iNKTs), and nivolumab at 200 mg every 14 days.

In some embodiments, administration of iNKT cells or a composition thereof (e.g., agenT-797), and optionally in combination with an ICI therapy, initiates cytokine (e.g., cytotoxic cytokine) release. In some embodiments, administration of iNKT cells or a composition thereof (e.g., agenT-797) increases cytotoxic cytokines (e.g., one or more of GZMB, GZMH, GZMA, GZMM, GNLY, FASLG, NKG7) in the subject by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 2 folds, at least 5 folds, at least 10 folds, at least 20 folds, at least 25 folds, at least 50 folds, at least 75 folds, at least 100 folds, at least 200 folds, at least 300 folds, at least 500 folds, at least 750 folds, at least 1000 folds, or more relative to the cytotoxic cytokine levels prior to administration. In some embodiments, administration of iNKT cells or a composition thereof (e.g., agenT-797), and optionally in combination with an ICI therapy, increases cytotoxic cytokines (e.g., one or more of GZMB, GZMH, GZMA, GZMM, GNLY, FASLG, NKG7) in the subject by the range of 1%-100%, 1%-90%, 1%-80%, 1%-70%, 1%-60%, 1%-70%, 1%-60%, 1%-50%, 1%-40%, 1%-30%, 1%-20%, 1%-10%, 1%-8%, 1%-5%, 1%-3%, 1%-2%, 5%-100%, 5%-90%, 5%-80%, 5%-70%, 5%-60%, 55%-50%, 5%-40%, 5%-30%, 5%-20%, 5%-10%, 5%-8%, 10%-100%, 10%-90%, 10%-80%, 10%-70%, 10%-60%, 10%-50%, 10%-40%, 10%-30%, 10%-20%, 20%-100%, 20%-90%, 20%-80%, 20%-70%, 20%-60%, 20%-50%, 20%-40%, 20%-30%, 30%-100%, 30%-90%, 30%-80%, 30%-70%, 30%-60%, 30%-50%, 30%-40%, 40%-100%, 40%-90%, 40%-80%, 40%-70%, 40%-60%, 40%-50%, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, 90%-100%, 1-1000 folds, 1-750 folds, 1-500 folds, 1-100 folds, 1-50 folds, 1-25 folds, 1-20 folds, 1-10 folds, 1-5 folds, 1-4 folds, 1-3 folds, 1-2 folds, 2-1000 folds, 2-750 folds, 2-500 folds, 2-100 folds, 2-50 folds, 2-25 folds, 2-20 folds, 2-10 folds, 2-5 folds, 5-1000 folds, 5-750 folds, 5-500 folds, 5-100 folds, 5-50 folds, 5-25 folds, 5-20 folds, 5-15 folds, 5-10 folds, 5-8 folds, 5-6 folds, 10-1000 folds, 10-750 folds, 10-500 folds, 10-100 folds, 10-50 folds, 10-25 folds, 10-20 folds, 10-15 folds, 20-1000 folds, 20-750 folds, 20-500 folds, 20-100 folds, 20-50 folds, 20-25 folds, 50-1000 folds, 50-750 folds, 50-500 folds, 50-100 folds, 50-75 folds, 100-1000 folds, 100-750 folds, 100-500 folds, or 100-250 folds relative to the cytotoxic cytokine levels prior to administration.

In some embodiments, administration of iNKT cells or a composition thereof (e.g., agenT-797), and optionally in combination with an ICI therapy, reduces tumor burden (e.g., tumor burden as measured by size, location and/or weight of tumor) by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% relative to tumor burden prior to administration. In some embodiments, administration of iNKT cells or a composition thereof (e.g., agenT-797), and optionally in combination with an ICI therapy, reduces tumor burden (e.g., tumor burden as measured by size, location and/or weight of tumor) by the range of 1%-100%, 1%-90%, 1%-80%, 1%-70%, 1%-60%, 1%-70%, 1%-60%, 1%-50%, 1%-40%, 1%-30%, 1%-20%, 1%-10%, 1%-8%, 1%-5%, 1%-3%, 1%-2%, 5%-100%, 5%-90%, 5%-80%, 5%-70%, 5%-60%, 55%-50%, 5%-40%, 5%-30%, 5%-20%, 5%-10%, 5%-8%, 10%-100%, 10%-90%, 10%-80%, 10%-70%, 10%-60%, 10%-50%, 10%-40%, 10%-30%, 10%-20%, 20%-100%, 20%-90%, 20%-80%, 20%-70%, 20%-60%, 20%-50%, 20%-40%, 20%-30%, 30%-100%, 30%-90%, 30%-80%, 30%-70%, 30%-60%, 30%-50%, 30%-40%, 40%-100%, 40%-90%, 40%-80%, 40%-70%, 40%-60%, 40%-50%, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% relative to tumor burden prior to administration.

In some embodiments, administration of iNKT cells or a composition thereof (e.g., agenT-797), and optionally in combination with an ICI therapy, reduces tumor metastasis by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% relative to metastasis prior to administration or to a subject having the same cancer at the same stage but did not receive the iNKT cells. In some embodiments, In some embodiments, administration of iNKT cells or a composition thereof (e.g., agenT-797), and optionally in combination with an ICI therapy, reduces metastasis by the range of 1%-100%, 1%-90%, 1%-80%, 1%-70%, 1%-60%, 1%-70%, 1%-60%, 1%-50%, 1%-40%, 1%-30%, 1%-20%, 1%-10%, 1%-8%, 1%-5%, 1%-3%, 1%-2%, 5%-100%, 5%-90%, 5%-80%, 5%-70%, 5%-60%, 55%-50%, 5%-40%, 5%-30%, 5%-20%, 5%-10%, 5%-8%, 10%-100%, 10%-90%, 10%-80%, 10%-70%, 10%-60%, 10%-50%, 10%-40%, 10%-30%, 10%-20%, 20%-100%, 20%-90%, 20%-80%, 20%-70%, 20%-60%, 20%-50%, 20%-40%, 20%-30%, 30%-100%, 30%-90%, 30%-80%, 30%-70%, 30%-60%, 30%-50%, 30%-40%, 40%-100%, 40%-90%, 40%-80%, 40%-70%, 40%-60%, 40%-50%, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% relative to metastasis prior to administration or to a subject having the same cancer at the same stage but did not receive the iNKT cells.

In some embodiments, administration of iNKT cells or a composition thereof (e.g., agenT-797), and optionally in combination with an ICI therapy, increases cytotoxic immune cell (e.g., CD8+ T cell, NK cell and/or iNKT cell) infiltration to the tumor by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 2 folds, at least 5 folds, at least 10 folds, at least 20 folds, at least 25 folds, at least 50 folds, at least 75 folds, at least 100 folds, at least 200 folds, at least 300 folds, at least 500 folds, at least 750 folds, at least 1000 folds, or more relative to the infiltrated cytotoxic immune cell level prior to administration. In some embodiments, administration of iNKT cells or a composition thereof (e.g., agenT-797), and optionally in combination with an ICI therapy, increases cytotoxic immune cell (e.g., CD8+ T cell, NK cell and/or iNKT cell) infiltration to the tumor by the range of 1%-100%, 1%-90%, 1%-80%, 1%-70%, 1%-60%, 1%-70%, 1%-60%, 1%-50%, 1%-40%, 1%-30%, 1%-20%, 1%-10%, 1%-8%, 1%-5%, 1%-3%, 1%-2%, 5%-100%, 5%-90%, 5%-80%, 5%-70%, 5%-60%, 55%-50%, 5%-40%, 5%-30%, 5%-20%, 5%-10%, 5%-8%, 10%-100%, 10%-90%, 10%-80%, 10%-70%, 10%-60%, 10%-50%, 10%-40%, 10%-30%, 10%-20%, 20%-100%, 20%-90%, 20%-80%, 20%-70%, 20%-60%, 20%-50%, 20%-40%, 20%-30%, 30%-100%, 30%-90%, 30%-80%, 30%-70%, 30%-60%, 30%-50%, 30%-40%, 40%-100%, 40%-90%, 40%-80%, 40%-70%, 40%-60%, 40%-50%, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, 90%-100%, 1-1000 folds, 1-750 folds, 1-500 folds, 1-100 folds, 1-50 folds, 1-25 folds, 1-20 folds, 1-10 folds, 1-5 folds, 1-4 folds, 1-3 folds, 1-2 folds, 2-1000 folds, 2-750 folds, 2-500 folds, 2-100 folds, 2-50 folds, 2-25 folds, 2-20 folds, 2-10 folds, 2-5 folds, 5-1000 folds, 5-750 folds, 5-500 folds, 5-100 folds, 5-50 folds, 5-25 folds, 5-20 folds, 5-15 folds, 5-10 folds, 5-8 folds, 5-6 folds, 10-1000 folds, 10-750 folds, 10-500 folds, 10-100 folds, 10-50 folds, 10-25 folds, 10-20 folds, 10-15 folds, 20-1000 folds, 20-750 folds, 20-500 folds, 20-100 folds, 20-50 folds, 20-25 folds, 50-1000 folds, 50-750 folds, 50-500 folds, 50-100 folds, 50-75 folds, 100-1000 folds, 100-750 folds, 100-500 folds, or 100-250 folds relative to the infiltrated cytotoxic immune cell level prior to administration.

In some embodiments, administration of iNKT cells or a composition thereof (e.g., agenT-797), and optionally in combination with an ICI therapy, increases T cell clonality (e.g., increased clonal expansion of T cells targeting neoantigens that previously exist in the TME but not able to undergo effective clonal expansion) in the tumor by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 2 folds, at least 5 folds, at least 10 folds, at least 20 folds, at least 25 folds, at least 50 folds, at least 75 folds, at least 100 folds, at least 200 folds, at least 300 folds, at least 500 folds, at least 750 folds, at least 1000 folds, or more relative to the T cell clonality level prior to administration. In some embodiments, administration of iNKT cells or a composition thereof (e.g., agenT-797), and optionally in combination with an ICI therapy, increases T cell clonality (e.g., increased clonal expansion of T cells targeting neoantigens that previously exist in the TME but not able to undergo effective clonal expansion) in the tumor by the range of 1%-100%, 1%-90%, 1%-80%, 1%-70%, 1%-60%, 1%-70%, 1%-60%, 1%-50%, 1%-40%, 1%-30%, 1%-20%, 1%-10%, 1%-8%, 1%-5%, 1%-3%, 1%-2%, 5%-100%, 5%-90%, 5%-80%, 5%-70%, 5%-60%, 55%-50%, 5%-40%, 5%-30%, 5%-20%, 5%-10%, 5%-8%, 10%-100%, 10%-90%, 10%-80%, 10%-70%, 10%-60%, 10%-50%, 10%-40%, 10%-30%, 10%-20%, 20%-100%, 20%-90%, 20%-80%, 20%-70%, 20%-60%, 20%-50%, 20%-40%, 20%-30%, 30%-100%, 30%-90%, 30%-80%, 30%-70%, 30%-60%, 30%-50%, 30%-40%, 40%-100%, 40%-90%, 40%-80%, 40%-70%, 40%-60%, 40%-50%, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, 90%-100%, 1-1000 folds, 1-750 folds, 1-500 folds, 1-100 folds, 1-50 folds, 1-25 folds, 1-20 folds, 1-10 folds, 1-5 folds, 1-4 folds, 1-3 folds, 1-2 folds, 2-1000 folds, 2-750 folds, 2-500 folds, 2-100 folds, 2-50 folds, 2-25 folds, 2-20 folds, 2-10 folds, 2-5 folds, 5-1000 folds, 5-750 folds, 5-500 folds, 5-100 folds, 5-50 folds, 5-25 folds, 5-20 folds, 5-15 folds, 5-10 folds, 5-8 folds, 5-6 folds, 10-1000 folds, 10-750 folds, 10-500 folds, 10-100 folds, 10-50 folds, 10-25 folds, 10-20 folds, 10-15 folds, 20-1000 folds, 20-750 folds, 20-500 folds, 20-100 folds, 20-50 folds, 20-25 folds, 50-1000 folds, 50-750 folds, 50-500 folds, 50-100 folds, 50-75 folds, 100-1000 folds, 100-750 folds, 100-500 folds, or 100-250 folds relative to the T cell clonality prior to administration.

In some embodiments, administration of iNKT cells or a composition thereof (e.g., agenT-797), and optionally in combination with an ICI therapy, increases intra-tumoral T cell expansion by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 2 folds, at least 5 folds, at least 10 folds, at least 20 folds, at least 25 folds, at least 50 folds, at least 75 folds, at least 100 folds, at least 200 folds, at least 300 folds, at least 500 folds, at least 750 folds, at least 1000 folds, or more relative to intra-tumoral T cell expansion prior to administration. In some embodiments, administration of iNKT cells or a composition thereof (e.g., agenT-797), and optionally in combination with an ICI therapy, increases intra-tumoral T cell expansion by the range of 1%-100%, 1%-90%, 1%-80%, 1%-70%, 1%-60%, 1%-70%, 1%-60%, 1%-50%, 1%-40%, 1%-30%, 1%-20%, 1%-10%, 1%-8%, 1%-5%, 1%-3%, 1%-2%, 5%-100%, 5%-90%, 5%-80%, 5%-70%, 5%-60%, 55%-50%, 5%-40%, 5%-30%, 5%-20%, 5%-10%, 5%-8%, 10%-100%, 10%-90%, 10%-80%, 10%-70%, 10%-60%, 10%-50%, 10%-40%, 10%-30%, 10%-20%, 20%-100%, 20%-90%, 20%-80%, 20%-70%, 20%-60%, 20%-50%, 20%-40%, 20%-30%, 30%-100%, 30%-90%, 30%-80%, 30%-70%, 30%-60%, 30%-50%, 30%-40%, 40%-100%, 40%-90%, 40%-80%, 40%-70%, 40%-60%, 40%-50%, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, 90%-100%, 1-1000 folds, 1-750 folds, 1-500 folds, 1-100 folds, 1-50 folds, 1-25 folds, 1-20 folds, 1-10 folds, 1-5 folds, 1-4 folds, 1-3 folds, 1-2 folds, 2-1000 folds, 2-750 folds, 2-500 folds, 2-100 folds, 2-50 folds, 2-25 folds, 2-20 folds, 2-10 folds, 2-5 folds, 5-1000 folds, 5-750 folds, 5-500 folds, 5-100 folds, 5-50 folds, 5-25 folds, 5-20 folds, 5-15 folds, 5-10 folds, 5-8 folds, 5-6 folds, 10-1000 folds, 10-750 folds, 10-500 folds, 10-100 folds, 10-50 folds, 10-25 folds, 10-20 folds, 10-15 folds, 20-1000 folds, 20-750 folds, 20-500 folds, 20-100 folds, 20-50 folds, 20-25 folds, 50-1000 folds, 50-750 folds, 50-500 folds, 50-100 folds, 50-75 folds, 100-1000 folds, 100-750 folds, 100-500 folds, or 100-250 folds relative to intra-tumoral T cell expansion prior to administration.

In some embodiments, administration of iNKT cells or a composition thereof (e.g., agenT-797), and optionally in combination with an ICI therapy, increases T cell activation (e.g., as evidenced by increased expression of T cell activation cytokines such as CD25, CD69, 4-1BB, HLA-DRA, CD44, and/or TCF1) by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 2 folds, at least 5 folds, at least 10 folds, at least 20 folds, at least 25 folds, at least 50 folds, at least 75 folds, at least 100 folds, at least 200 folds, at least 300 folds, at least 500 folds, at least 750 folds, at least 1000 folds, or more relative to T cell activation prior to administration. In some embodiments, administration of iNKT cells or a composition thereof (e.g., agenT-797), and optionally in combination with an ICI therapy, increases T cell activation (e.g., as evidenced by increased expression of T cell activation cytokines such as CD25, CD69, 4-1BB, HLA-DRA, CD44, and/or TCF1) by the range of 1%-100%, 1%-90%, 1%-80%, 1%-70%, 1%-60%, 1%-70%, 1%-60%, 1%-50%, 1%-40%, 1%-30%, 1%-20%, 1%-10%, 1%-8%, 1%-5%, 1%-3%, 1%-2%, 5%-100%, 5%-90%, 5%-80%, 5%-70%, 5%-60%, 55%-50%, 5%-40%, 5%-30%, 5%-20%, 5%-10%, 5%-8%, 10%-100%, 10%-90%, 10%-80%, 10%-70%, 10%-60%, 10%-50%, 10%-40%, 10%-30%, 10%-20%, 20%-100%, 20%-90%, 20%-80%, 20%-70%, 20%-60%, 20%-50%, 20%-40%, 20%-30%, 30%-100%, 30%-90%, 30%-80%, 30%-70%, 30%-60%, 30%-50%, 30%-40%, 40%-100%, 40%-90%, 40%-80%, 40%-70%, 40%-60%, 40%-50%, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, 90%-100%, 1-1000 folds, 1-750 folds, 1-500 folds, 1-100 folds, 1-50 folds, 1-25 folds, 1-20 folds, 1-10 folds, 1-5 folds, 1-4 folds, 1-3 folds, 1-2 folds, 2-1000 folds, 2-750 folds, 2-500 folds, 2-100 folds, 2-50 folds, 2-25 folds, 2-20 folds, 2-10 folds, 2-5 folds, 5-1000 folds, 5-750 folds, 5-500 folds, 5-100 folds, 5-50 folds, 5-25 folds, 5-20 folds, 5-15 folds, 5-10 folds, 5-8 folds, 5-6 folds, 10-1000 folds, 10-750 folds, 10-500 folds, 10-100 folds, 10-50 folds, 10-25 folds, 10-20 folds, 10-15 folds, 20-1000 folds, 20-750 folds, 20-500 folds, 20-100 folds, 20-50 folds, 20-25 folds, 50-1000 folds, 50-750 folds, 50-500 folds, 50-100 folds, 50-75 folds, 100-1000 folds, 100-750 folds, 100-500 folds, or 100-250 folds relative to T cell activation prior to administration.

In some embodiments, administration of iNKT cells or a composition thereof (e.g., agenT-797), and optionally in combination with an ICI therapy, increases Th1 polarization (e.g., as evidenced by increased expression of TBET, EOMES, CCL3, CCL4, IFNG, and/or TNF) by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 2 folds, at least 5 folds, at least 10 folds, at least 20 folds, at least 25 folds, at least 50 folds, at least 75 folds, at least 100 folds, at least 200 folds, at least 300 folds, at least 500 folds, at least 750 folds, at least 1000 folds, or more relative to Th1 polarization prior to administration. In some embodiments, administration of iNKT cells or a composition thereof (e.g., agenT-797), and optionally in combination with an ICI therapy, increases Th1 polarization (e.g., as evidenced by increased expression of TBET, EOMES, CCL3, CCL4, IFNG, and/or TNF) by the range of 1%-100%, 1%-90%, 1%-80%, 1%-70%, 1%-60%, 1%-70%, 1%-60%, 1%-50%, 1%-40%, 1%-30%, 1%-20%, 1%-10%, 1%-8%, 1%-5%, 1%-3%, 1%-2%, 5%-100%, 5%-90%, 5%-80%, 5%-70%, 5%-60%, 55%-50%, 5%-40%, 5%-30%, 5%-20%, 5%-10%, 5%-8%, 10%-100%, 10%-90%, 10%-80%, 10%-70%, 10%-60%, 10%-50%, 10%-40%, 10%-30%, 10%-20%, 20%-100%, 20%-90%, 20%-80%, 20%-70%, 20%-60%, 20%-50%, 20%-40%, 20%-30%, 30%-100%, 30%-90%, 30%-80%, 30%-70%, 30%-60%, 30%-50%, 30%-40%, 40%-100%, 40%-90%, 40%-80%, 40%-70%, 40%-60%, 40%-50%, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, 90%-100%, 1-1000 folds, 1-750 folds, 1-500 folds, 1-100 folds, 1-50 folds, 1-25 folds, 1-20 folds, 1-10 folds, 1-5 folds, 1-4 folds, 1-3 folds, 1-2 folds, 2-1000 folds, 2-750 folds, 2-500 folds, 2-100 folds, 2-50 folds, 2-25 folds, 2-20 folds, 2-10 folds, 2-5 folds, 5-1000 folds, 5-750 folds, 5-500 folds, 5-100 folds, 5-50 folds, 5-25 folds, 5-20 folds, 5-15 folds, 5-10 folds, 5-8 folds, 5-6 folds, 10-1000 folds, 10-750 folds, 10-500 folds, 10-100 folds, 10-50 folds, 10-25 folds, 10-20 folds, 10-15 folds, 20-1000 folds, 20-750 folds, 20-500 folds, 20-100 folds, 20-50 folds, 20-25 folds, 50-1000 folds, 50-750 folds, 50-500 folds, 50-100 folds, 50-75 folds, 100-1000 folds, 100-750 folds, 100-500 folds, or 100-250 folds relative to Th1 polarization prior to administration.

In some embodiments, administration of iNKT cells or a composition thereof (e.g., agenT-797), and optionally in combination with an ICI therapy, increases NK cell (e.g., as evidenced by increased expression of NK cell receptors such as DNAM1, 2B4, SLAMF6, NKG2C, NKG2A, and/or CD56) in the tumor by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 2 folds, at least 5 folds, at least 10 folds, at least 20 folds, at least 25 folds, at least 50 folds, at least 75 folds, at least 100 folds, at least 200 folds, at least 300 folds, at least 500 folds, at least 750 folds, at least 1000 folds, or more relative to NK cell prior to administration. In some embodiments, administration of iNKT cells or a composition thereof (e.g., agenT-797), and optionally in combination with an ICI therapy, increases NK cell (e.g., as evidenced by increased expression of NK cell receptors such as DNAM1, 2B4, SLAMF6, NKG2C, NKG2A, and/or CD56) in the tumor by the range of 1%-100%, 1%-90%, 1%-80%, 1%-70%, 1%-60%, 1%-70%, 1%-60%, 1%-50%, 1%-40%, 1%-30%, 1%-20%, 1%-10%, 1%-8%, 1%-5%, 1%-3%, 1%-2%, 5%-100%, 5%-90%, 5%-80%, 5%-70%, 5%-60%, 55%-50%, 5%-40%, 5%-30%, 5%-20%, 5%-10%, 5%-8%, 10%-100%, 10%-90%, 10%-80%, 10%-70%, 10%-60%, 10%-50%, 10%-40%, 10%-30%, 10%-20%, 20%-100%, 20%-90%, 20%-80%, 20%-70%, 20%-60%, 20%-50%, 20%-40%, 20%-30%, 30%-100%, 30%-90%, 30%-80%, 30%-70%, 30%-60%, 30%-50%, 30%-40%, 40%-100%, 40%-90%, 40%-80%, 40%-70%, 40%-60%, 40%-50%, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, 90%-100%, 1-1000 folds, 1-750 folds, 1-500 folds, 1-100 folds, 1-50 folds, 1-25 folds, 1-20 folds, 1-10 folds, 1-5 folds, 1-4 folds, 1-3 folds, 1-2 folds, 2-1000 folds, 2-750 folds, 2-500 folds, 2-100 folds, 2-50 folds, 2-25 folds, 2-20 folds, 2-10 folds, 2-5 folds, 5-1000 folds, 5-750 folds, 5-500 folds, 5-100 folds, 5-50 folds, 5-25 folds, 5-20 folds, 5-15 folds, 5-10 folds, 5-8 folds, 5-6 folds, 10-1000 folds, 10-750 folds, 10-500 folds, 10-100 folds, 10-50 folds, 10-25 folds, 10-20 folds, 10-15 folds, 20-1000 folds, 20-750 folds, 20-500 folds, 20-100 folds, 20-50 folds, 20-25 folds, 50-1000 folds, 50-750 folds, 50-500 folds, 50-100 folds, 50-75 folds, 100-1000 folds, 100-750 folds, 100-500 folds, or 100-250 folds relative to NK cells prior to administration.

In some embodiments, administration of iNKT cells or a composition thereof (e.g., agenT-797), and optionally in combination with an ICI therapy, increases expression level of one or more of genes (e.g., GZMB, GZMH, GZMA, GZMK, GZMM, GNLY, PRF1, LEF1, TRAIL, FASLG, LAMP1, NKG7, CD25, CD69, 4-1BB, HLA-DRA, CD44, TCF1, TBET, EOMES, CCL3, CCL4, IFNG, TNF, DNAM1, 2B4, SLAMF6, NKG2D, NKG2C, NKG2A, or CD56) by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 2 folds, at least 5 folds, at least 10 folds, at least 20 folds, at least 25 folds, at least 50 folds, at least 75 folds, at least 100 folds, at least 200 folds, at least 300 folds, at least 500 folds, at least 750 folds, at least 1000 folds, or more relative to expression level of these genes to administration. In some embodiments, administration of iNKT cells or a composition thereof (e.g., agenT-797), and optionally in combination with an ICI therapy, increases expression level of one or more of genes (e.g., GZMB, GZMH, GZMA, GZMK, GZMM, GNLY, PRF1, LEF1, TRAIL, FASLG, LAMP1, NKG7, CD25, CD69, 4-1BB, HLA-DRA, CD44, TCF1, TBET, EOMES, CCL3, CCL4, IFNG, TNF, DNAM1, 2B4, SLAMF6, NKG2D, NKG2C, NKG2A, or CD56) by the range of 1%-100%, 1%-90%, 1%-80%, 1%-70%, 1%-60%, 1%-70%, 1%-60%, 1%-50%, 1%-40%, 1%-30%, 1%-20%, 1%-10%, 1%-8%, 1%-5%, 1%-3%, 1%-2%, 5%-100%, 5%-90%, 5%-80%, 5%-70%, 5%-60%, 55%-50%, 5%-40%, 5%-30%, 5%-20%, 5%-10%, 5%-8%, 10%-100%, 10%-90%, 10%-80%, 10%-70%, 10%-60%, 10%-50%, 10%-40%, 10%-30%, 10%-20%, 20%-100%, 20%-90%, 20%-80%, 20%-70%, 20%-60%, 20%-50%, 20%-40%, 20%-30%, 30%-100%, 30%-90%, 30%-80%, 30%-70%, 30%-60%, 30%-50%, 30%-40%, 40%-100%, 40%-90%, 40%-80%, 40%-70%, 40%-60%, 40%-50%, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, 90%-100%, 1-1000 folds, 1-750 folds, 1-500 folds, 1-100 folds, 1-50 folds, 1-25 folds, 1-20 folds, 1-10 folds, 1-5 folds, 1-4 folds, 1-3 folds, 1-2 folds, 2-1000 folds, 2-750 folds, 2-500 folds, 2-100 folds, 2-50 folds, 2-25 folds, 2-20 folds, 2-10 folds, 2-5 folds, 5-1000 folds, 5-750 folds, 5-500 folds, 5-100 folds, 5-50 folds, 5-25 folds, 5-20 folds, 5-15 folds, 5-10 folds, 5-8 folds, 5-6 folds, 10-1000 folds, 10-750 folds, 10-500 folds, 10-100 folds, 10-50 folds, 10-25 folds, 10-20 folds, 10-15 folds, 20-1000 folds, 20-750 folds, 20-500 folds, 20-100 folds, 20-50 folds, 20-25 folds, 50-1000 folds, 50-750 folds, 50-500 folds, 50-100 folds, 50-75 folds, 100-1000 folds, 100-750 folds, 100-500 folds, or 100-250 folds relative to expression level of these genes prior to administration.

II. Unmodified, Allogeneic Invariant Natural Killer T (iNKT) Cells

In some aspects, the present disclosure provides a composition comprising invariant natural killer T (iNKT) cells. The term “invariant Natural Killer T cells”, or “invariant NKT cells”, “iNKT cells”, or “Type I NKT cell), as used herein, refer to a population of T lymphocytes expressing a conserved semi-invariant TCR specific for lipid antigens restricted for the monomorphic MHC class I-related molecule CD1d. Natural killer T cells (NKT cells) were originally characterized in mice as T cells that express both a TCR and NK1.1 (NKR-Pla-c or CD161), a C-type lectin NK receptor. Invariant NKT (iNKT) cells express a semi-invariant αβ TCR (e.g., formed by an invariant TRAV11-TRAJ18 (4) rearrangement in mice, or the homologous invariant TRAV10-TRAJ18 chain in humans), paired with a limited set of diverse Vβ chains, predominantly TRBV1, TRBV29, or TRBV13 in mice (6) and TRBV25 in humans (see e.g., Dellabona et al., An invariant V alpha 24-J alpha Q/V beta 11 T cell receptor is expressed in all individuals by clonally expanded CD4-8-T cells. J Exp Med. (1994) 180:1171-6. 10.1084). The semi-invariant TCR recognizes exogenous and endogenous lipid antigens presented by the monomorphic MHC class I-related molecule CD1d (see e.g., Brennan et al., Invariant natural killer T cells: an innate activation scheme linked to diverse effector functions. Nat Rev Immunol. (2013) 13:101-17. 10.1038). Exogenous lipid antigens include the prototypical α-Galactosylceramide (α-GalCer) (Kawano et al., CD1d-restricted and TCR-mediated activation of valpha14 NKT cells by glycosylceramides. Science. (1997) 278:1626-9. 10.1126) and a number of bacterial-derived Ags, which can activate iNKT cells.

iNKT cells undergo a distinct developmental pathway compared to T cells, leading to the acquisition of innate effector functions already in the thymus. Thymic iNKT cells indeed express markers usually upregulated by peripheral effector/memory T cells, such as CD44 and CD69, together with distinctive NK differentiation markers, such as NK1.1 (in some mouse genetic backgrounds, CD161 in humans), CD122 (the IL-2R/IL-15R β-chain), CD94/NKG2 and Ly49 (A-J), and a broad spectrum of TH1/2/17 effector cytokines. Once migrated in the periphery, iNKT cells form a tissue resident population that survey the cellular integrity and rapidly respond to local damage and inflammation, jump starting the reaction by cells of the innate and adaptive immune response.

Because iNKT cells can rapidly produce IFNγ, IL-4, or both, they have been found to play a role in various diseases by establishing a context-dependent Th1- or Th2-based immune response. In bacterial and viral infections, iNKT cells typically help in early control of the pathogen by establishing a productive Th1 response. In both mouse and human studies, roles for iNKT cells have been described in diseases associated with excessive Th1 responses like type 1 diabetes and chronic obstructive pulmonary disease. Roles have also been described for iNKT cells helping to suppress Th1 responses and drive tolerogenic responses to grafts. As an example, following hematopoietic stem cell transfer, the presence of iNKT cells is predictive for survival with a reduction in graft versus host disease (GvHD) in patients and preclinical models.

Accordingly, the present disclosure provides a composition comprising iNKT cells. iNKT cell therapy can be autologous, allogeneic or xenogeneic. In some embodiments, the subject and the donor are allogeneic. The term “allogeneic”, as used herein, is a word denoting tissue and/or cells taken from different individuals of the same species, and the tissue and/or cells are genetically dissimilar and immunologically incompatible. iNKT cells, being restricted for the monomorphic CD1d molecule, are substantially devoid of alloreactivity, allowing them to be used off-the-shelf in a donor-unrestricted manner (e.g., without causing graft-versus-host disease (GvHD)).

In some aspects, the present disclosure also provides methods for preparing a population of immune cells (e.g., iNKT cells composition such as agenT-797). An initial population of iNKT cells can be obtained from any source, such as peripheral blood mononuclear cells (PBMCs), bone marrow, tissues such as spleen, lymph node, thymus, or tumor tissue. A source suitable for obtaining the type of cell desired would be evident to one of skill in the art. In some embodiments, the population of iNKT cells in one composition (e.g., one dose of an agenT-797 composition) is initially isolated from PBMCs (e.g., from one single donor or two or more donors) and expanded ex vivo. In some embodiments, a blood sample or an apheresis is taken from a generally healthy subject (e.g., a donor). In some embodiments, a blood sample or an apheresis is taken from a generally healthy subject who is at risk of developing a disease, but who has not yet developed a disease (e.g., a donor), and the cells of interest are isolated and frozen for later use. In some embodiments, the iNKT cells may be expanded, frozen, and used at a later time.

In some embodiments, the iNKT cells are isolated (e.g., purified or enriched) from peripheral blood mononuclear cells (PBMCs) from apheresis of the donor. In some embodiments, the isolated iNKT cells are expanded ex vivo. In some embodiments, an initial population of the iNKT cells are purified from PBMCs using a suitable method known in the art (e.g., FACS or MACS). In some embodiments, the initial population of the iNKT cells are stimulated by α-galactosylceramide (α-GalCer) or any modified glycolipid thereof, e.g., as described by Zhang et al., α-GalCer and iNKT Cell-Based Cancer Immunotherapy: Realizing the Therapeutic Potentials, Front Immunol. 2019 Jun. 6; 10:1126; Schafer et al., iNKT cell stimulation by glycolipid ligands modified from α-galactosylceramide results in differential interleukin-2 secretion profiles, J Immunol May 1, 2019, 202 (1 Supplement) 177.1) for activation and expansion. In some embodiments, the initial population of the iNKT cells are stimulated by α-GalCer while being co-cultured with PBMCs. In some embodiments, the PBMCs are irradiated prior to being co-cultured with iNKT cells. In some embodiments, the PBMCs are pulsed with α-GalCer. In some embodiments, the stimulation of iNKT cells with α-GalCer and PBMCs (e.g., irradiated PBMCs) In some embodiments, the iNKT cells can go through more than one round of stimulation as described herein. After expansion, at least 70%, at least 75%, at least 80%, at least 85%, at least 90%, at least 91%, at least 92%, at least 93%, at least 94%, at least 95%, at least 96%, at least 97%, at least 98%, at least 99%, or 100% of the cells in the composition are iNKT cells. In some embodiments, the iNKT cells are unmodified (e.g., not genetically modified to express exogenous genes). The present disclosure also contemplates the use of iNKT cells isolated and/or expanded using any suitable known methods in the art.

In some embodiments, expanded, unmodified iNKT cells express both Th1 type cytokines (e.g., IFNγ, TNFα, GM-CSF) and Th2 type cytokines (e.g., IL-4, IL-13). In some embodiments, after expansion, the iNKT cells retain their inherent cytotoxic capacity against CD1d-expressing cells. In some embodiments, the iNKT cell therapy composition is agenT-797.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials. In some embodiments, compositions of the present disclosure are formulated for intravenous administration.

Without further elaboration, it is believed that one skilled in the art can, based on the above description, utilize the present disclosure to its fullest extent. Particular embodiments are, therefore, to be construed as merely illustrative, and not limitative of the remainder of the disclosure in any way whatsoever.

### EXAMPLES

The following examples are provided for illustrative purposes and are not intended to limit the scope of the disclosure.

### Example 1: Invariant Natural Killer T (iNKT) Cell Anti-Cancer Mechanisms

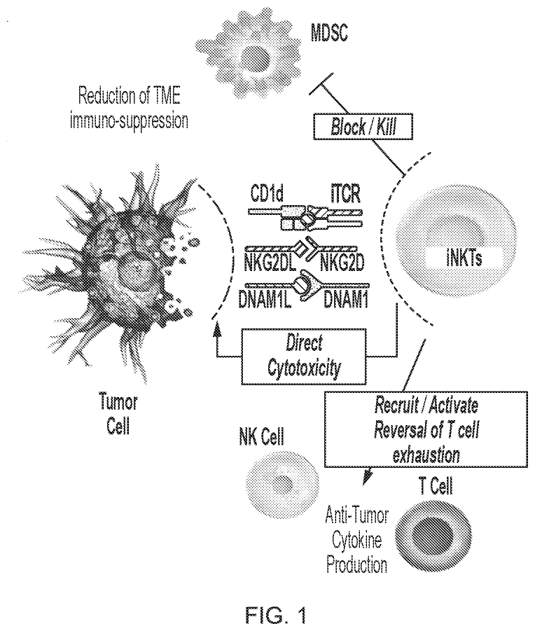

iNKT cells directly recognize and kill tumor cells through TCR-dependent recognition of CD1d ligands on tumor cells or through recognition of stress signals through NK receptors NKG2D and DNAM1. Further, iNKT cells promoter anti-tumor responses within the tumor microenvironment (TME) by: 1) promoting anti-inflammatory M1 macrophages and killing tolerogenic M2 macrophages, 2) Relieving immunosuppression in the TME by suppression or killing of MDSCs, or 3) Recruitment and activation of other tumor-targeting immune cells such as NK cells and cytotoxic T cells (CTL) through interferon-gamma (IFN-γ) and interleukin-2 (IL-2) (FIG. 1).

### Example 2: Phase 1/2 Trial Evaluating Safety and Efficacy of Allogeneic iNKT Cells, AgenT-797, in Patients with Advanced or Metastatic Solid Tumors

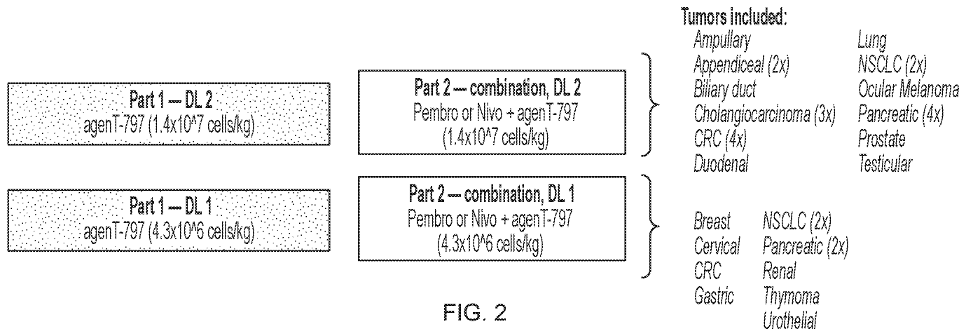

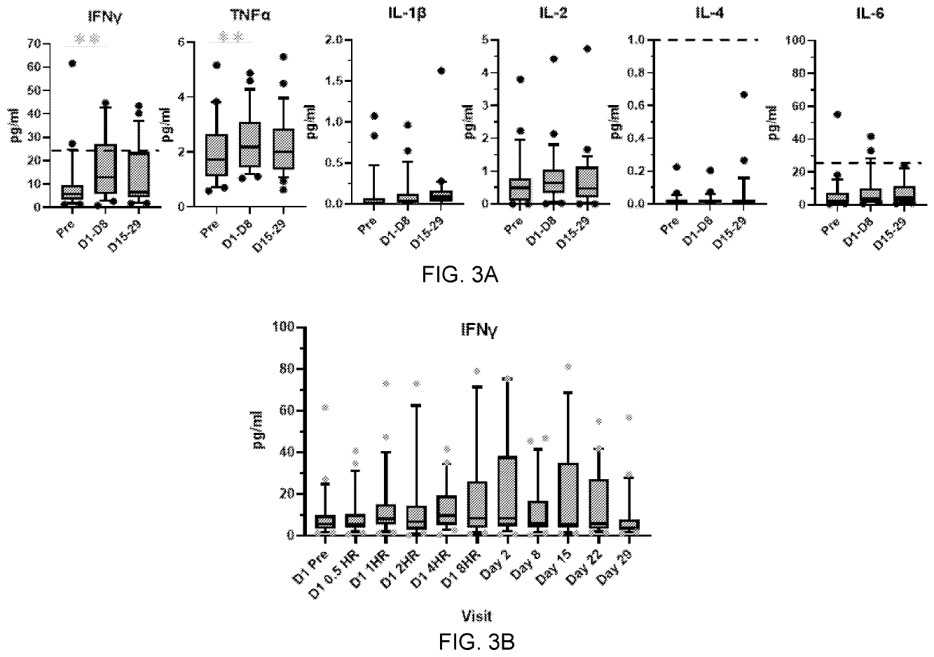

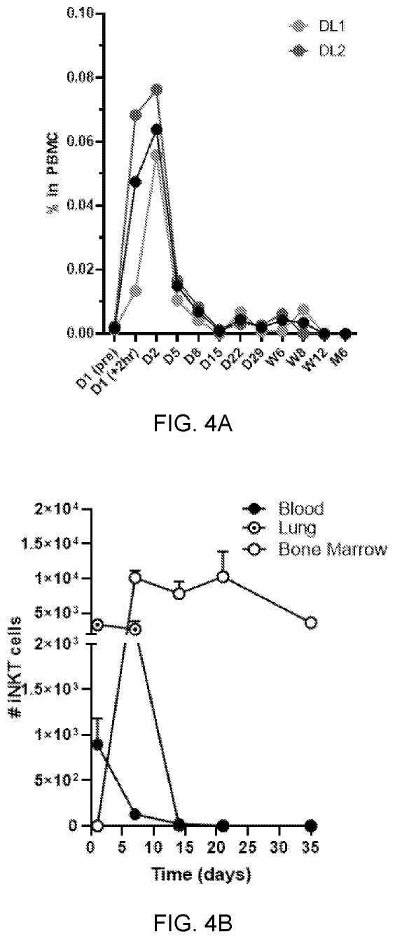

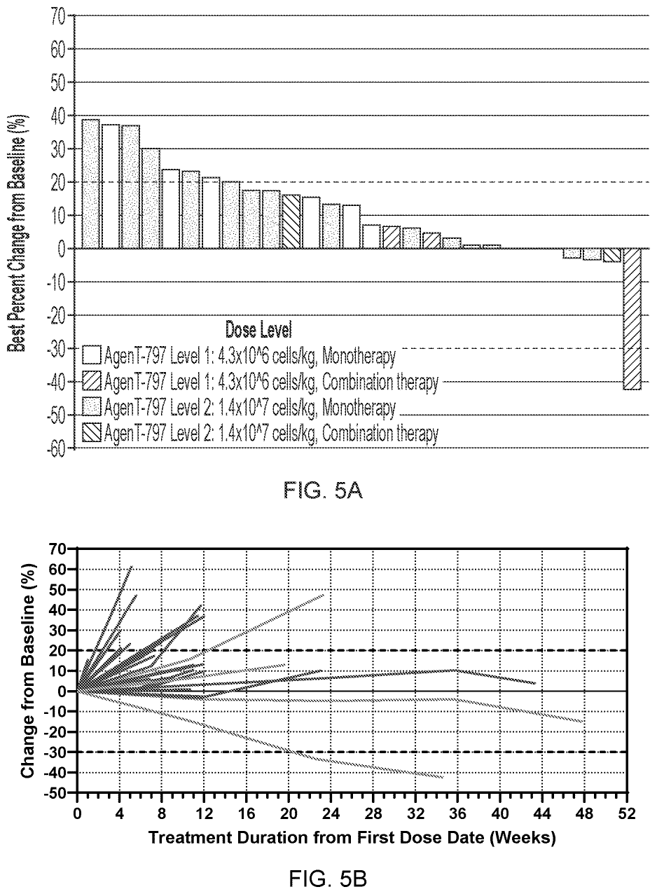

Patients were selected based on certain inclusion criteria. Individuals had a confirmed diagnosis of advanced or metastatic solid tumor that was refractory to standard therapy or for which no standard therapy was available. Patients with prior anti-CD1d monoclonal antibody treatment or active autoimmune disease requiring systemic treatment were excluded. Additionally, patients had measurable disease on imaging based on RECIST 1.1. As endpoints, the safety, persistence of agenT-797, and anti-tumor activity were measured as duration of response, progression-free survival, and time to response. The treatment was a 3+3 dose escalation of agenT-797 administered by single intravenous infusion. An overview of the study design is shown in FIG. 2.

### Example 3: AgenT-797 was Well Tolerated in Patients Across Multiple Tumor Types

Following treatment, the most common Treatment Adverse Event (TRAE) was fatigue (n=5) and the most severe TRAE (grade 3) was anemia (n=1). No grade ≥3 neurotoxicity or cytokine release syndrome (CRS) was observed. agenT-797 was well tolerated at all dose levels tested and no MTD was determined. Table 2 shows a summary of AEs.

TABLE 1

| | | agenT-797 | agenT-797 + | agenT-797 | agenT-797 + |
|---|---|---|---|---|---|
| | | MonoTx | anti-PD-1 | MonoTx | anti-PD-1 |
| | Total | (DL1) | (DL1) | (DL2) | (DL2) |
| | Dose level | | | | |
| | | 4.3 × 10⁶ | 4.3 × 10⁶ | 1.4 × 10⁷ | 1.4 × 10⁷ |
| | | cells/kg | cells/kg | cells/kg | cells/kg |
| | Subjects dosed (n) | | | | |
| | 34 | 8 | 3 | 20 | 3 |
| | n (%) | n (%) | n (%) | n (%) | n (%) |
| AE | 32 (94.1) | 8 (100) | 3 (100) | 18 (90.0) | 3 (100) |
| Any AE of | 19 (55.9) | 7 (87.5) | 0 | 11 (55.0) | 1 (33.3) |
| grade ≥3 | | | | | |
| irTEAE | 3 (8.8) | 0 | 0 | 2 (10.0) | 1 (33.3) |
| Any irTEAE of | 1 (2.9) | 0 | 0 | 0 | 1 (33.3) |
| grade ≥3 | | | | | |
| TRAE | 9 (26.5) | 3 (37.5) | 2 (66.7) | 2 (10.0) | 2 (66.7) |
| Any TRAE of | 1 (2.9) | 1 (12.5) | 0 | 0 | 0 |
| grade ≥3 | | | | | |
| Any TRAE | 0 | 0 | 0 | 0 | 0 |
| leading to | | | | | |
| discontinuation | | | | | |
| Any TRAE | 0 | 0 | 0 | 0 | 0 |
| leading to dose | | | | | |
| interruption | | | | | |
| Any TRAE | 0 | 0 | 0 | 0 | 0 |
| leading to death | | | | | |
| TRAE by | | | | | |
| System Organ | | | | | |
| Class | | | | | |
| General (Fatigue, | 5 (14.7) | 1 (12.5) | 1 (33.3) | 1 (5.0) | 2 (66.7) |
| Chills) | | | | | |
| Skin (Pruritus, | 2 (5.9) | 1 (12.5) | 1 (33.3) | 0 | 0 |
| Odour) | | | | | |
| Immune system | 1 (2.9) | 0 | 0 | 1 (5.0) | 0 |
| (CRS) | | | | | |
| Nervous system | 1 (2.9) | 0 | 0 | 0 | 1 (33.3) |
| (Dysgeusia) | | | | | |
| Psychiatric | 1 (2.9) | 0 | 1 (33.3) | 0 | 0 |
| (Insomnia) | | | | | |
| Respiratory, | 1 (2.9) | 0 | 0 | 1 (5.0) | 0 |
| thoracic, | | | | | |
| mediastinal | | | | | |
| (Dyspnoea) | | | | | |
| Blood and | 1 (2.9) | 1 (12.5) | 0 | 0 | 0 |
| lymphatic system | | | | | |
| (Anemia) | | | | | |

During the course of treatment, no DLTs were reported by the subjects treated. TEAEs were observed in 32/34 subjects (94.1%). The most common TEAEs were fatigue (n=16), nausea (n=8) and constipation (n=4) as well as peripheral oedema, abdominal distension, and dyspnea (n=6 each). TEAEs of grade ≥3 were experienced by a total of 19 subjects, largely consistent with disease progression. One subject (DL2, combination therapy) experienced an immune-related TEAEs (irTEAE) of grade ≥3 (skin rash, grade 3), likely as a result of anti-PD treatment. One subject (DL1, monotherapy) experienced a TRAE of grade 3 (anemia).

### Example 4: Biomarker Response in Specific Cancer Types

The biomarker response in PD-1 refractory testicular cancer showed a 41% AFP reduction at 9 months; response ongoing. The patient for this study was a 49 year old male with five failed prior therapies: 1) Bleomycin+etoposide+cisplatin (CR), 2) Vinblastine+ifosfamise+cisplatin+pembrolizumab (PR), 3) Etoposide+carboplatin+autologous stem cell transplant (PR), 4) Etoposide+ipilimumab+nivolumab (PD), 5) CD1D RM 684 (PD). A single dose of agent-797+nivolumab (200 mg) was administered at a does level of 1.4×10⁷ cells. The baseline AFP response was 5.18 ng/ml and at three months the AFP was 3.06 ng/mL. The AFP response and stable disease are ongoing.

Testicular Cancer Patient

| | |
|---|---|
| Patient | 49-year-old male |
| Characteristics | Failed 5 prior therapies |
| Prior therapies | Bleomycin + etoposide + cisplatin CR |
| | Vinblastine + ifosfamise + cisplatin + pembrolizumab |
| | PR |
| | Etoposide + carboplatin + autologous stem cell |
| | transplant PR |
| | Etoposide + ipilimumab + nivolumab PD |
| | CD1D RM 684 PD |
| Treatment | Single dose of agent-797 + nivolumab (200 mg) |
| | DL2: 1.4 × 101 cells |
| Response | Baseline AFP: 5.18 ng/mL |
| | AFP at 3 months: 3.06 ng/mL |
| | AFP response and stable disease ongoing |

### Example 5: Monotherapy and Combination Therapy

The agenT-797 alone and in combination with anti-PD-1 without lymphodepletion was well tolerated across multiple doses 60% DCR (2SD, 1PR) observed in 5 evaluable patients treated in combination with checkpoint inhibitors. agenT-797 persisted in the periphery for up to 8 weeks. agenT-797 demonstrated systemic and local proinflammatory TH-1 signatures and increased tumor infiltration by cytotoxic lymphocytes (NK cells and CTL). Early signals of clinical and biomarker activity were observed in heavily pre-treated patients who progressed on prior checkpoint inhibitors. agenT-797 may drive clonal T cell expansion in cancers with high neoantigen burden. Translational data were consistent with iNKT cells to enable T cell and NK cell trafficking to tumors, reinvigorate partially exhausted CD8+ T cells, and improve effector functions within the tumor microenvironment. Observations of a proinflammatory signature associated with agenT-797 in the treatment of solid tumors contrasts with the use of agenT-797 in severe COVID19/ARDS (NCT04582201), where an anti-inflammatory signature dominates, suggesting a plasticity of the agenT-797 mediated response in relation to the specific disease context. The Phase 1/2 study will be expanded into PD-1 refractory NSCLC and advanced gastric cancer to further investigate the cells in combination with immune-checkpoint-inhibitors.

### Example 6. Overcoming Resistance to Programmed Cell Death Protein 1 (PD-1) Blockade with Allogeneic Invariant Natural Killer T-Cells (iNKT)

Gastric cancer is the fifth most common malignancy worldwide and accounts for nearly 700,000 deaths yearly [1]. Early-stage disease is treated with multi-modal therapy: systemic chemotherapy, radiation, and surgical resection [2]. Metastatic gastric cancer is treated primarily with palliative chemotherapy alone or combined with immune checkpoint inhibitors such as nivolumab. The median overall survival (OS) of patients with metastatic gastric cancer receiving chemotherapy alone ranges from 6 to 14 months [3]. The outcomes of patients have improved with the incorporation of anti-PD-1 nivolumab [4-6]. However, tumor response to ICI blockade is limited to approximately 12% of gastric cancer patients [7]. Tumor responses are higher (30-60%) among the 20-25% of gastric cancers harboring microsatellite instability. Nevertheless, most patients with gastric cancer treated with ICI will develop primary or acquired resistance to checkpoint blockade [8]. Novel therapeutic strategies to overcome these resistance mechanisms are urgently needed to optimize the clinical management of these patients.

Invariant Natural Killer T (iNKT) cells are a subset of T cells that have emerged as a promising therapeutic strategy [9]. iNKT cells initiate and propagate cytokine release in response to recognizing lipid antigens, coupling the innate and adaptive immune responses. Investigational allogenic iNKT cell infusions have entered clinical stage development with promising preliminary results [9]. agenT-797 is an off-the-shelf cell therapy comprising >95% allogenic human unmodified iNKT cells isolated from one healthy donor mononuclear cell apheresis unit and expanded ex vivo. Clinical studies evaluating agenT-797 in solid tumors commenced in early 2022 (NCT05108623). Presented here is a case of a patient with MSI-H gastric cancer with acquired resistance to anti-PD-1 nivolumab who enrolled in a Phase I/II trial investigating agenT-797 in patients with advanced solid tumors. The patient achieved a confirmed partial response per RECIST and remained on treatment for eight months before developing progressive disease of the primary gastric tumor.

### Case Presentation

A 73-year-old male presented to his primary care physician for a routine follow-up where he was found to have iron deficiency anemia. Endoscopy revealed a large, ulcerated, non-circumferential mass on the lesser curvature of the stomach. Biopsy showed a moderately differentiated, HER-2 negative adenocarcinoma. Tumor loss of MLH-1 and PMS-2 characterized the tumor as MSI-H. A computed tomography (CT) scan of the chest/abdomen/pelvis showed a heterogeneous, ulcerated, and partially necrotic gastric fundal mass consistent with the visualized mass on endoscopy and associated peritoneal carcinomatosis.

### Treatment Course

The patient was initially treated with 4 cycles of pembrolizumab monotherapy. Subsequent CT scan showed increased size of peritoneal nodules. Given ongoing transfusion-dependent anemia, the patient had a repeat endoscopy (EGD), demonstrating progression and increased size of the previously visualized gastric mass with associated bleeding. Treatment was modified to nivolumab with folinic acid, 5-fluorouracil and oxaliplatin (FOL-FOX). A repeat EGD performed three months later revealed a significant reduction in tumor size, and CT scan showed stable metastatic peritoneal disease. After 22 treatments (17 months), restaging CT scan demonstrated worsening of the proximal gastric wall thickening and progression of an adjacent omental nodularity. Biopsy of the enlarged nodule confirmed moderately differentiated adenocarcinoma consistent with the known gastric primary malignancy. The tumor was HER-2 negative and PD-L1 positive (combined positivity score [CPS] score 7) by immunohistochemical (IHC) assay. IHC also revealed loss of both MLH-1 and PSM-2 nuclear expression with intact expression of MSH-2 and MSH-6 characterizing the tumor as having high microsatellite instability (MSI-H).

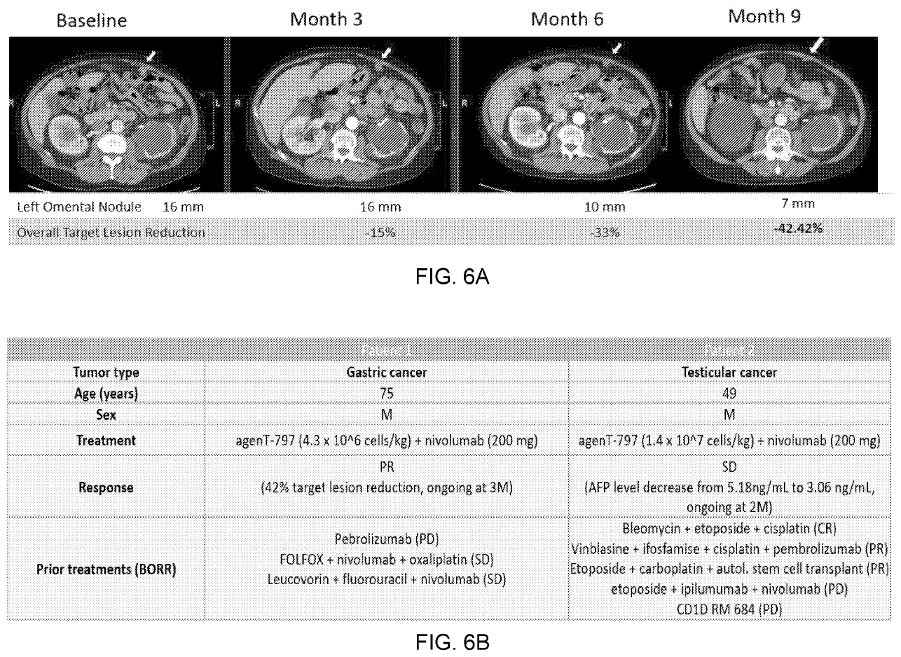

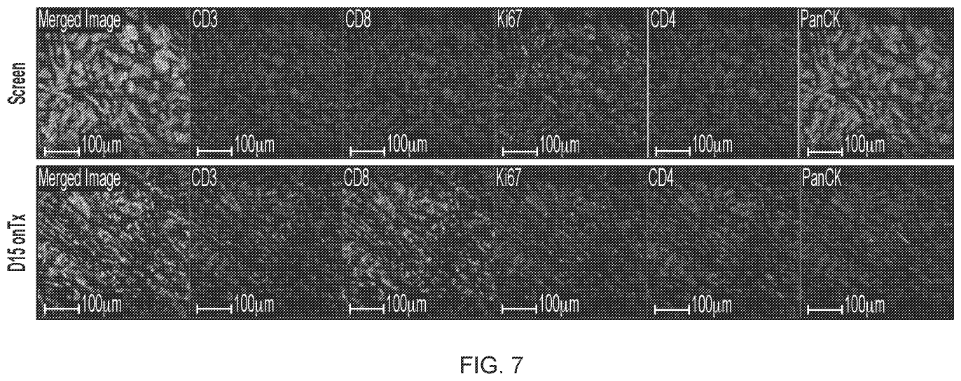

Two months after evidence of disease progression, the patient enrolled in a Phase I clinical trial evaluating agenT-797 (Allogeneic Invariant Natural Killer T (iNKT) Non-Transduced Cells) with and without the addition of ICI. The patient received one infusion of agenT-797 (4.3×106 cells/kg) and continued nivolumab maintenance (200 mg every 14 days). CT scans performed after one month of treatment revealed stable thickening of the stomach wall with interval decreased size of omental nodularity. During treatment with nivolumab, the patient developed a grade 2 rash attributed to nivolumab, which improved with topical steroids. The patient did not experience any other immune-related adverse effects while on trial. CT scans performed at 3, 6, and 9 months (6, 12, and 18 cycles of nivolumab, respectively) demonstrated a further decrease in the size of the omental nodules (FIG. 6A).

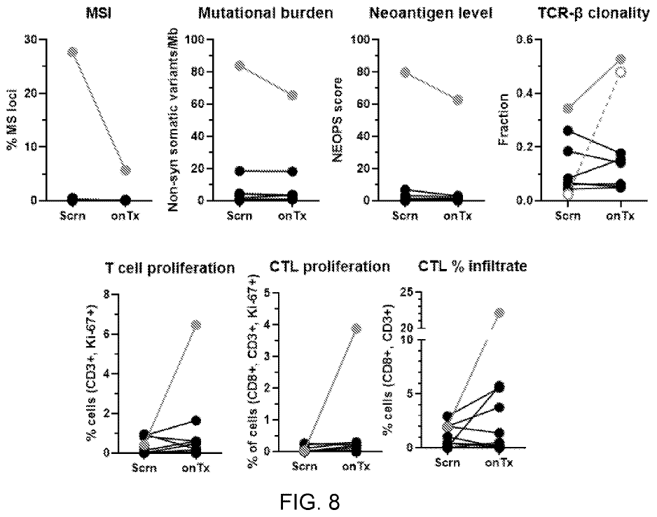

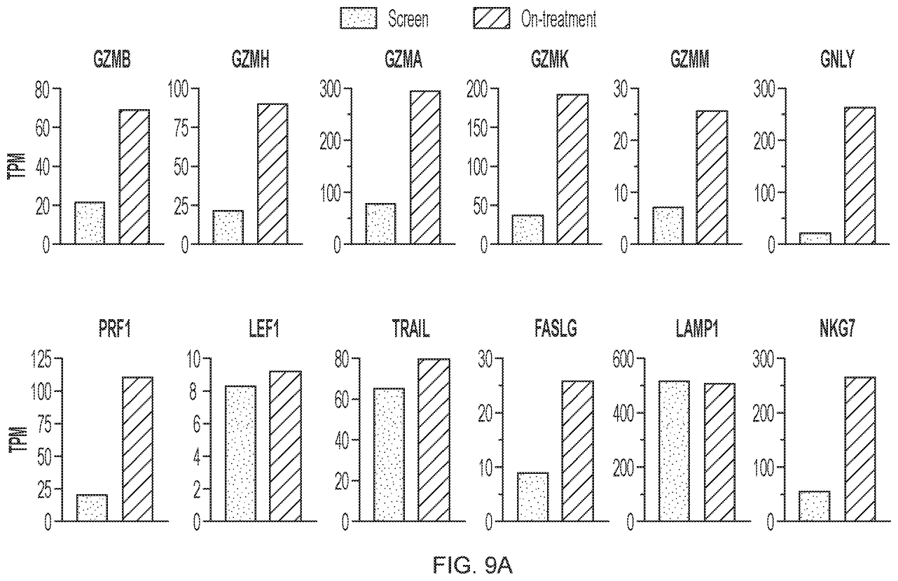

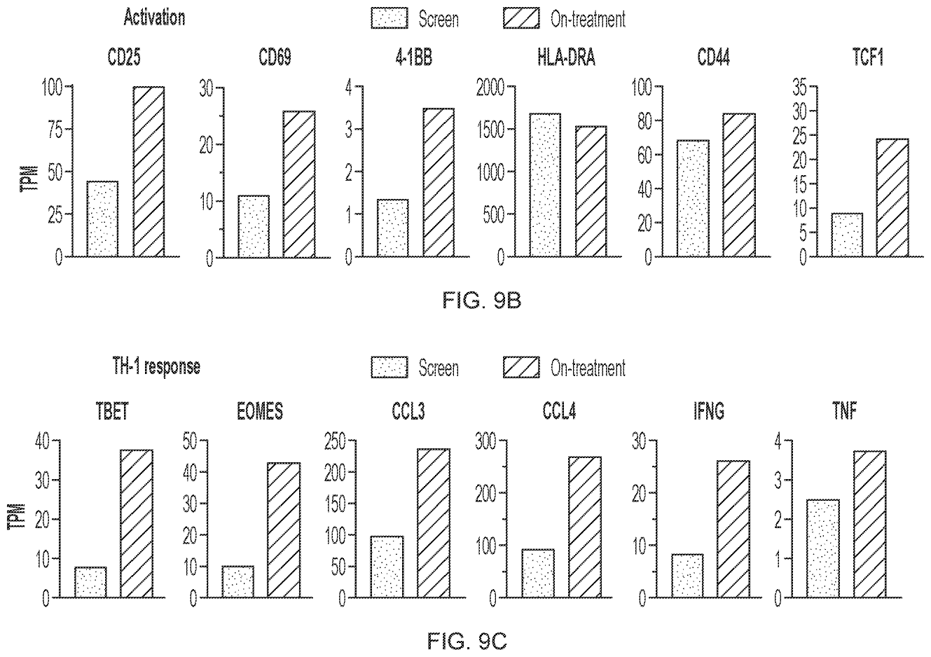

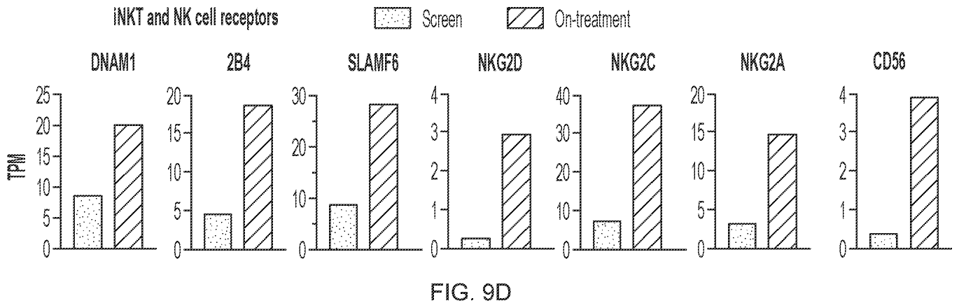

Whole exome sequencing (WES) of baseline and on-treatment (Day 15) tumor biopsy specimens confirmed the tumor to be MSI-H with a high mutational burden of 84 mut/MB (FIG. 8, MSI and Mutational burden). High mutational burden correlated with a high neoantigen score (FIG. 8, Neoantigen level). T-cell receptor (TCR) sequence analysis of RNA sequencing (RNAseq) data on tumor material indicated that tumor-infiltrating T cells were highly clonal (FIG. 8, TCR-β clonality). T cell clonality increased following treatment with agenT-797. The baseline tumor sample prior to study revealed mutations in antigen presentation including JAK2 and TAF8 (RNA allelic fraction of 0.09 and 0.43, respectively). At day 15, JAK2 allelic fraction had increased to 0.13, while TAF8 had decreased to 0.22. This analysis revealed a post-treatment switch in the predominant TCR clone, with a marked expansion of a minority pre-existent T cell clone (expanding from 2 to 48%) following the infusion of agenT-797 (FIG. 8, TCR-β clonality, open circles, dotted line). Multiplex immunofluorescence staining and automated image analysis of tumor FFPE sections indicated extensive tumor infiltration by CD8+ T cells following treatment with agenT-797 (FIG. 7 and FIG. 8, CTL infiltrate). Markers of cellular proliferation indicated intra-tumoral expansion of T cells (FIG. 8, T cell proliferation and CTL proliferation). Gene expression analysis of paired tumor biopsies at baseline and on-treatment (D15) showed increased immune infiltration, activation, and TH-1 polarization following infusion of agenT-797 (FIGS. 9A-9D). These signatures are consistent with the infiltration of tumor by cytotoxic lymphocytes, including CD8+ T cells, NK cells, or iNKT cells.

Taken together, these data suggest that this patient's high tumor mutational burden led to increased neoantigen presentation, which was only partially effective at driving clonal expansion of anti-tumor T cells, even in the presence of anti-PD-1 therapy. Administration of agenT-797 appeared to overcome the patient's resistance to anti-PD-1 therapy, leading to tumor immune infiltration of TH-1 lymphocytes, unblocking of neoantigen-driven clonal expansion, and activation of anti-tumor cytotoxic T lymphocytes (CTLs). After 11 cycles of treatment, worsening of gastric wall thickening was observed on CT scan, and endoscopy confirmed the progression of the primary gastric tumor. The patient was taken off the study.

### Discussion

Presented herein is a case of a patient with MSI-H gastric adenocarcinoma with resistance to the PD-1 inhibitor, nivolumab, with a durable confirmed response by RECIST 1.1 criteria following treatment with investigational iNKT cell therapy in combination with anti-PD-1 therapy.

Patients with primary resistance to immunotherapy fail to achieve a response to initial treatment. Several proposed mechanisms lead to primary immunotherapeutic resistance, including lack of cancer neo-antigens and mutations within the antigen-presenting MHC proteins, resulting in a failure to prime T-cells [10]. Acquired resistance occurs in patients who have achieved a temporary response to checkpoint blockade followed by evidence of disease progression [11]. Mechanisms of acquired resistance include changes in the tumor expression of neoantigens and alterations within the tumor microenvironment (TME), compromising T-lymphocyte cytotoxic effects. Of note, WES of tumor biopsies from this patient identified numerous mutations potentially impacting innate and adaptive immunity, including a missense mutation in the gene encoding Janus Kinase 2 (JAK2). Alterations in the Janus Kinase (JAK) 1/2 interferon-gamma signaling pathway have been implicated in the development of acquired resistance to checkpoint inhibitors [12].

Loss of function mutations within the JAK 1/2 pathway results in absent interferon-gamma (IFN-γ) production, a central cytokine in evoking the innate immune response. Downregulated innate immunity plays a significant role in primary and acquired immune checkpoint inhibitor resistance [13].

The patient presented in this case had no response to initial therapy with pembrolizumab, indicating primary resistance to single-agent PD-1 therapy. He was then treated with a combination of anti-PD-1 therapy and cytotoxic chemotherapy, which resulted in a durable response of 17 months. In the CheckMate-649 trial, adding nivolumab to chemotherapy improved the median overall survival (mOS) to 13.1 months compared to 11.1 months with chemotherapy alone [5]. The patient demonstrated a significantly better response than expected with chemotherapy alone, supporting the additive effect of anti-PD-1 therapy with eventually acquired resistance.

Therapeutic strategies, including adoptive cell transfer (ACT), invoking the innate immune response have garnered significant interest in overcoming both primary and acquired resistance to checkpoint blockade. Pre-clinical studies have demonstrated that iNKT cells are integral in initiating and propagating an anti-tumor immune response in solid tumor malignancies [14, 15].

Harnessing the immune-evoking potential of iNKT cells to augment existing immunotherapeutic strategies has emerged as a potential treatment strategy. iNKT cells, a subset of T cells with properties of innate immunity, induce tumor-killing effects through a multitude of actions, including direct cytotoxic effects on tumor cells as well as promotion of anti-tumor immune response by recruitment of other T cell populations as well as cytokines and chemokines. iNKT cells exert their anti-tumor activity mainly by two mechanisms. The first is the direct activation of the iNKT T-cell receptor (TCR) through recognition of endogenous tumor lipid antigens or exogenous α-Galactosylceramide both of which activate iNKT cells and simulate IFN-γ leading to direct cytotoxic effects [16]. A second downstream effect of increased IFN-γ production is the maturation of dendritic cells (DCs). Maturation of DCs leads to the production of interleukin-12 (IL-12) that stimulates further production of IFN-γ resulting in recruitment and activation of both NK and T-cells. Moreover, it has been demonstrated that iNKT cells can reverse the exhaustion of CD8+ T cells (manuscript under review) typically observed in the TME, thus reinvigorating the endogenous immune response. The recruitment and activation of T-cells within the tumor microenvironment (TME) leads to continued tumor recognition and direct cytotoxic effects [17, 18].

In this case, pre- and post-treatment biopsies of the metastatic gastric nodules demonstrated increased immune cell infiltration correlating with the observed radiographic partial response (FIGS. 7-8). Post-treatment tissue analyzed with multiplex immunofluorescent staining demonstrated increases in T-cell proliferation, cytotoxic T-cell (CTL) infiltration, and proliferation. RNA sequencing gene analysis was also performed, which demonstrated increased levels of several cytotoxic cytokines, makers of T-Helper 1 (TH1) response, and NK cell activation. These post-treatment assays support an agenT-797-driven immune response contributing to the patient's partial and durable radiographic response on treatment. Though direct tumor infiltration by agenT-797 iNKT cells awaits confirmation (due to the current lack of iNKT-specific reagents suitable for staining FFPE sections), the observed hallmarks of iNKT activity within tumor tissue, including IFN-γ upregulation and tumor infiltration by cytotoxic lymphocytes, strongly suggest the presence and activity of agenT-797 within the tumor. Unfortunately, after 11 months of treatment, the patient experienced disease progression and was taken off the study. There have been multiple proposed mechanisms for acquired resistance to iNKT cells, including dysregulated trafficking of NK cells into the TME, downregulation of neoantigens by cancerous cells, down-regulated MHC-1 cell surface proteins in response to TME-related hypoxia as well as immunosuppressive signaling from within the TME impairing NK cell functioning [19-24]. Initial results of the Phase 1 trial, which evaluated agenT-797, alone or in combination with pembrolizumab or nivolumab in patients with advanced solid tumors, demonstrated an overall rate of response (ORR) of 20% with a favorable adverse event profile [25].

### Methods

agenT-797 manufacture: agent-797 is a single donor, ex-vivo expanded allogeneic iNKT cell therapy product. iNKT expansion is largely as previously described. Briefly, iNKT cells are isolated from healthy donor leukapheresis by microbead-bound monoclonal antibody to the invariant TCR of iNKT cells. Isolated iNKT cells undergo two rounds of stimulation by irradiated PBMC pulsed with the iNKT-specific ligand αGalCer over several weeks. Expanded iNKT cells undergo harvesting, formulation, aseptic filling and cryopreservation. Purity of manufactured iNKT cell products measured >95%. Manufacturing of agent-797 is unbiased towards any iNKT subtype, and expanded iNKT cells retain the broad cytokine secretion profile upon stimulation which is also observed in iNKT cells freshly isolated from PBMC. agenT-797 is manufactured under GMP at the Cell Manipulation Core facility at the Dana Faber Cancer Institute (Boston, MA) and at MINK Therapeutics (Lexington, MA).

Biopsy Processing and Analysis: Fresh tumor biopsies from study NCT05108623 were embedded at PPD central laboratories (Highland Heights, KY) to generate FFPE tissue blocks. Blocks were sent to Precision for Medicine (Houston, TX) for sectioning and analysis by multiplex immunofluorescence (mIF). mIF utilized a qualified Opal multiplex antibody panel against markers for T cells (CD3, CD4, CD8), cell activation (Ki-67), and cancer cells (Pan-CK), as well as DAPI for staining nuclei. Slides were imaged using the Akoya Vectra Polaris fully automated digital pathology system (Akoya Biosciences) and evaluated by auto-mated image analysis using HALO imaging software (Indica Labs). FFPE blocks were further sent to Personalis (Fremont, CA) for nucleic acid extraction and analysis by whole exome sequencing (WES) and RNA sequencing (RNAseq). Sequencing data was analyzed using Personalis' ImmunoID NeXT Analytics platform.

### REFERENCES

1. Rawla P, Barsouk A. Epidemiology of gastric cancer: global trends, risk factors and prevention. Prz Gastroenterol. 2019; 14:26-38.
2. Lordick F, Carneiro F, Cascinu S, Fleitas T, Haustermans K, Piessen G, et al. Gastric cancer: ESMO Clinical Practice Guideline for diagnosis, treatment and follow-up. Ann Oncol. 2022; 33:1005-20.
3. Hu H M, Tsai H J, Ku H Y, Lo S S, Shan Y S, Chang H C, et al. Survival outcomes of management in metastatic gastric adenocarcinoma patients. Sci Rep. 2021; 11:23142.
4. Janjigian Y Y, Kawazoe A, Yanez P, Li N, Lonardi S, Kolesnik O, et al. The KEYNOTE-811 trial of dual PD-1 and HER2 blockade in HER2-positive gastric cancer. Nature. 2021; 600:727-30.
5. Janjigian Y Y, Shitara K, Moehler M, Garrido M, Salman P, Shen L, et al. First-line nivolumab plus chemotherapy versus chemotherapy alone for advanced gastric, gastro-oesophageal junction, and oesophageal adenocarcinoma (CheckMate 649): a randomised, open-label, phase 3 trial. Lancet. 2021; 398:27-40.
6. Kang Y K, Boku N, Satoh T, Ryu M H, Chao Y, Kato K, et al. Nivolumab in patients with advanced gastric or gastro-oesophageal junction cancer refractory to, or intolerant of, at least two previous chemotherapy regimens (ONO-4538-12, ATTRACTION-2): a randomised, double-blind, placebo-controlled, phase 3 trial. Lancet. 2017; 390:2461-71.
7. Kono K, Nakajima S, Mimura K. Current status of immune checkpoint inhibitors for gastric cancer. Gastric Cancer. 2020; 23:565-78.
8. Baxter M A, Middleton F, Cagney H P, Petty R D. Resistance to immune checkpoint inhibitors in advanced gastro-oesophageal cancers. Br J Cancer. 2021; 125:1068-79.
9. Ingram Z, Madan S, Merchant J, Carter Z, Gordon Z, Carey G, et al. Targeting Natural Killer T Cells in Solid Malignancies. Cells. 2021; 10:1329.
10. Said S S, Ibrahim W N. Cancer Resistance to Immunotherapy: Comprehensive Insights with Future Perspectives. Pharmaceutics. 2023; 15:1143.
11. Chen X, Zhang W, Yang W, Zhou M, Liu F. Acquired resistance for immune checkpoint inhibitors in cancer immunotherapy: challenges and prospects. Aging. 2022; 14:1048-64.
12. Zaretsky J M, Garcia-Diaz A, Shin D S, Escuin-Ordinas H, Hugo W, Hu-Lieskovan S, et al. Mutations Associated with Acquired Resistance to PD-1 Blockade in Mela-noma. N Engl J Med. 2016; 375:819-29.
13. Jenkins R W, Barbie D A, Flaherty K T. Mechanisms of resistance to immune checkpoint inhibitors. Br J Cancer. 2018; 118:9-16.
14. Wu L, Yun Z, Tagawa T, De la Maza L, Wu M O, Yu J, et al. Activation of CD1d-restricted natural killer T cells can inhibit cancer cell proliferation during che-motherapy by promoting the immune responses in murine mesothelioma. Cancer Immunol Immunother. 2014; 63:1285-96.
15. Shaked Y, Pham E, Hariharan S, Magidey K, Beyar-Katz O, Xu P, et al. Evidence Implicating Immunological Host Effects in the Efficacy of Metronomic Low-Dose Chemotherapy. Cancer Res. 2016; 76:5983-93.
16. Dhodapkar M V, Richter J. Harnessing natural killer T (NKT) cells in human mye-loma: progress and challenges. Clin Immunol. 2011; 140:160-6.
17. Liu D, Song L, Wei J, Courtney A N, Gao X, Marinova E, et al. IL-15 protects NKT cells from inhibition by tumor-associated macrophages and enhances antimetastatic activity. J Clin Invest. 2012; 122:2221-33. Oncogene (2024) 43:758-762
18. Metelitsa L S. Anti-tumor potential of type-I NKT cells against CD1d-positive and CD1d-negative tumors in humans. Clin Immunol. 2011; 140:119-29.
19. Sordo-Bahamonde C, Vitale M, Lorenzo-Herrero S, Lopez-Soto A, Gonzalez S. Mechanisms of Resistance to NK Cell Immunotherapy. Cancers. 2020; 12:893.
20. Nayyar G, Chu Y, Cairo M S. Overcoming Resistance to Natural Killer Cell Based Immunotherapies for Solid Tumors. Front Oncol. 2019; 9:51.
21. Gras Navarro A, Bjorklund A T, Chekenya M. Therapeutic potential and challenges of natural killer cells in treatment of solid tumors. Front Immunol. 2015; 6:202.
22. Melero I, Rouzaut A, Motz G T, Coukos G. T-cell and NK-cell infiltration into solid tumors: a key limiting factor for efficacious cancer immunotherapy. Cancer Dis-cov. 2014; 4:522-6.
23. Siemens D R, Hu N, Sheikhi A K, Chung E, Frederiksen L J, Pross H, et al. Hypoxia increases tumor cell shedding of MHC class I chain-related molecule: role of nitric oxide. Cancer Res. 2008; 68:4746-53.
24. Rusakiewicz S, Semeraro M, Sarabi M, Desbois M, Locher C, Mendez R, et al. Immune infiltrates are prognostic factors in localized gastrointestinal stromal tumors. Cancer Res. 2013; 73:3499-510.
25. Carneiro B, Garmezy B, Hamm J T, Sanborn R E, Wise-Draper T, Khoueiry A E-, et al. Phase 1 clinical update of allogeneic invariant natural killer T cells (iNKTs), agenT-797, alone or in combination with pembrolizumab or nivolumab in patients with advanced solid tumors. Cancer Res. 2023; 83: CT275-CT275.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms. Definition of terms are disclosed throughout the specification, including but not limited to the “Definition” section.

The section heads are not meant to be interpreted to limit the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A method for treating a relapsed/refractory solid tumor cancer, the method comprising administering to a subject in need thereof a composition comprising at least 95% allogeneic invariant natural killer cells (iNKTs).

2. The method of claim 1, wherein the iNKT cells are isolated from a single donor.

3. The method of claim 1, wherein the iNKT cells are isolated from two or more donors.

4. The method of any one of claims 1-3, wherein the relapsed/refractory solid tumor cancer is gastric cancer, lung cancer, or testicular cancer.

5. The method of any one of claims 1-4, wherein the relapsed/refractory solid tumor cancer is ampullary cancer, appendiceal cancer, biliary duct cancer, cholangiocarcinoma, colorectal cancer (CRC), duodenal cancer, non-small cell lung carcinoma (NSCLC), ocular melanoma, pancreatic cancer, or prostate cancer.

6. The method of any one of claims 1-5, wherein the subject has been previously administered one or more of the following agents: nivolumab, pembrolizumab, botensilimab, FOLFOX, oxaliplatin, leucovorin, fluorocil, bleomycin, etopidide, cisplatin, vinblasine, ifofamise, carboplatin, ipilumumab, CD1D RM 684, or an autologous stem cell transplant.

7. The method of any one of claims 1-6, wherein the subject has not previously been administered an anti-CD1d monoclonal antibody.

8. The method of any one of claims 1-7, wherein the has not undergone lymphodepletion prior to the administration.

9. The method of any one of claims 1-8, wherein the subject does not have an active autoimmune disorder.

10. The method of any one of claims 1-9, wherein from about 4.3×10⁶ to about 1.4×10⁷ iNKT cells/kg are administered to the subject.

11. The method of any one of claims 1-10, wherein the subject has gastric cancer and is administered about 4.3×10⁶ iNKT cells/kg.

12. The method of any one of claims 1-10, wherein the subject has testicular cancer and is administered about 1.4×10⁷ iNKT cells/kg.

13. The method of any one of claims 1-12, wherein the method further comprises administering one or more immune checkpoint inhibitors (ICI) to the subject.

14. The method of claim 13, wherein the one or more ICI comprises nivolumab, pembrolizumab, and/or botensilimab.

15. The method of claim 13 or 14, wherein the method comprises administering 200 mg of nivolumab to the subject.

16. The method of any one of claims 1-15, wherein after administration of the iNKTs to the subject, a level of one or more pro-inflammatory cytokines is increased relative to the level of the one or more pro-inflammatory cytokine prior to the administration.

17. The method of claim 16, wherein the one or more pro-inflammatory cytokine is IFNγ or TNFα.

18. The method of any one of claims 1-17, wherein after administration of the iNKTs to the subject, a level of immune infiltration of the subject's TME is increased relative to the level of immune infiltration of the TME prior to the administration.

19. The method of any one of claims 1-18, wherein after administration of the iNKTs to the subject, an expression level of one or more of the following genes is increased relative to the expression level of the one or more genes prior to the administration: GZMB, GZMH, GZMA, GZMK, GZMM, GNLY, PRF1, LEF1, TRAIL, FASLG, LAMP1, NKG7, CD25, CD69, 4-1BB, HLA-DRA, CD44, TCF1, TBET, EOMES, CCL3, CCL4, IFNG, TNF, DNAM1, 2B4, SLAMF6, NKG2D, NKG2C, NKG2A, or CD56.

20. The method of any one of claims 1-19, wherein administration of the iNKTs to the subject results in inhibition of growth or shrinkage of a tumor in the subject.

21. The method of any one of claims 1-20, wherein the subject is a human.

22. A method for treating a relapsed/refractory solid tumor cancer, the method comprising administering to a subject in need thereof:
(i) a composition comprising at least 95% allogeneic invariant natural killer cells (iNKTs); and
(ii) an immune checkpoint inhibitor (ICI).

23. The method of claim 22, wherein the iNKT cells are isolated from a single donor.

24. The method of claim 22, wherein the iNKT cells are isolated from two or more donors.

25. The method of any one of claims 22-24, wherein the relapsed/refractory solid tumor cancer is selected from gastric cancer, lung cancer, or testicular cancer.

26. The method of any one of claims 22-25, wherein the relapsed/refractory solid tumor cancer is selected from ampullary cancer, appendiceal cancer, biliary duct cancer, cholangiocarcinoma, colorectal cancer (CRC), duodenal cancer, non-small cell lung carcinoma (NSCLC), ocular melanoma, pancreatic cancer, and prostate cancer.

27. The method of any one of claims 22-26, wherein the subject has been previously administered one or more of the following agents: nivolumab, pembrolizumab, botensilimab, FOLFOX, oxaliplatin, leucovorin, fluorocil, bleomycin, etopidide, cisplatin, vinblasine, ifofamise, carboplatin, ipilumumab, CD1D RM 684, or an autologous stem cell transplant.

28. The method of any one of claims 22-27, wherein the subject has not previously been administered an anti-CD1d monoclonal antibody.

29. The method of any one of claims 22-28, wherein the administration does not comprise toxic lymphodepletion of the subject.

30. The method of any one of claims 22-29, wherein the subject does not have an active autoimmune disorder.

31. The method of any one of claims 22-30, wherein from about 4.3×10⁶ to about 1.4×10⁷ iNKT cells/kg are administered to the subject.

32. The method of any one of claims 22-31, wherein the subject has gastric cancer and is administered about 4.3×10⁶ iNKT cells/kg.

33. The method of any one of claims 22-31, wherein the subject has testicular cancer and is administered about 1.4×10⁷ iNKT cells/kg.

34. The method of any one of claims 22-33, wherein the ICI comprises nivolumab.

35. The method of claim 34, wherein the one or more ICI comprises pembrolizumab.

36. The method of claim 35, wherein 200 mg of nivolumab is administered to the subject.

37. The method of any one of claims 22-36, wherein after administration of the iNKTs to the subject, a level of one or more pro-inflammatory cytokines is increased relative to the level of the one or more pro-inflammatory cytokine prior to the administration.

38. The method of claim 37, wherein the one or more pro-inflammatory cytokine is IFNγ or TNFα.

39. The method of any one of claims 22-38, wherein after administration of the iNKTs to the subject, a level of immune infiltration of the subject's TME is increased relative to the level of immune infiltration of the TME prior to the administration.

40. The method of any one of claims 22-39, wherein after administration of the iNKTs to the subject, an expression level of one or more of the following genes is increased relative to the expression level of the one or more genes prior to the administration: GZMB, GZMH, GZMA, GZMK, GZMM, GNLY, PRF1, LEF1, TRAIL, FASLG, LAMP1, NKG7, CD25, CD69, 4-1BB, HLA-DRA, CD44, TCF1, TBET, EOMES, CCL3, CCL4, IFNG, TNF, DNAM1, 2B4, SLAMF6, NKG2D, NKG2C, NKG2A, or CD56.

41. The method of any one of claims 22-40, wherein administration of the iNKTs to the subject results in inhibition of growth or shrinkage of a tumor in the subject.

42. The method of any one of claims 22-41, wherein the subject is a human.

43. A method for treating gastric cancer in a subject in need thereof, the method comprising administering to the subject:
(i) a composition comprising at least 95% allogeneic invariant natural killer cells (iNKTs); and
(ii) an immune checkpoint inhibitor (ICI).

44. The method of claim 43, wherein the iNKT cells are isolated from a single donor.

45. The method of claim 43, wherein the iNKT cells are isolated from two or more donors.

46. The method of any one of claims 43-45, wherein the gastric cancer is high microsatellite instability (MSI-High) gastric cancer.

47. The method of any one of claims 43-46, wherein the gastric cancer is biliary duct cancer or cholangiocarcinoma.

48. The method of any one of claims 43-47, wherein the subject has been previously administered one or more of the following agents: nivolumab, pembrolizumab, botensilimab, FOLFOX, oxaliplatin, leucovorin, fluorocil, bleomycin, etopidide, cisplatin, vinblasine, ifofamise, carboplatin, ipilumumab, CD1D RM 684, or an autologous stem cell transplant.

49. The method of any one of claims 43-48, wherein the subject has not previously been administered an anti-CD1d monoclonal antibody.

50. The method of any one of claims 43-49, wherein the administration does not comprise toxic lymphodepletion of the subject.

51. The method of any one of claims 43-50, wherein the subject does not have an active autoimmune disorder.

52. The method of any one of claims 43-51, wherein from about 4.3×10⁶ to about 1000×10⁶ iNKT cells/kg are administered to the subject.

53. The method of any one of claims 43-52, wherein the subject has gastric cancer and is administered about 4.3×10⁶ iNKT cells/kg.

54. The method of any one of claims 43-53, wherein the ICI comprises a PD-1 inhibitor.

55. The method of any one of claims 43-54, wherein the PD-1 inhibitor comprises nivolumab.

56. The method of claim 55, wherein the PD-1 inhibitor comprises pembrolizumab.

57. The method of claim 55 or 56, wherein the method comprises administering 200 mg of nivolumab to the subject.

58. The method of any one of claims 43-57, wherein after administration of the iNKTs to the subject, a level of one or more pro-inflammatory cytokines is increased relative to the level of the one or more pro-inflammatory cytokine prior to the administration.

59. The method of claim 58, wherein the one or more pro-inflammatory cytokine is IFNγ or TNFα.

60. The method of any one of claims 43-59, wherein after administration of the iNKTs to the subject, a level of immune infiltration of the subject's TME is increased relative to the level of immune infiltration of the TME prior to the administration.

61. The method of any one of claims 43-60, wherein after administration of the iNKTs to the subject, an expression level of one or more of the following genes is increased relative to the expression level of the one or more genes prior to the administration: GZMB, GZMH, GZMA, GZMK, GZMM, GNLY, PRF1, LEF1, TRAIL, FASLG, LAMP1, NKG7, CD25, CD69, 4-1BB, HLA-DRA, CD44, TCF1, TBET, EOMES, CCL3, CCL4, IFNG, TNF, DNAM1, 2B4, SLAMF6, NKG2D, NKG2C, NKG2A, or CD56.

62. The method of any one of claims 43-61, wherein administration of the iNKTs to the subject results in inhibition of growth or shrinkage of a tumor in the subject.

63. The method of any one of claims 43-62, wherein the subject is a human.
